(19) United States
(12) Patent Application Publication
(10) Pub. No.: US 2023/0393041 A1
(43) Pub. Date: Dec. 7, 2023

(54) Bead-Enabled, Efficient, and Rapid Multi-Omic Sample Preparation for Mass Spectrometry Analysis

(71) Applicant: Wisconsin Alumni Research Foundation, Madison, WI (US)

(72) Inventors: Joshua COON, Middleton, WI (US); Laura MUEHLBAUER, Madison, WI (US); Yunyun ZHU, Madison, WI (US); Katherine OVERMYER, Madison, WI (US); Annie JEN, Madison, WI (US)

(73) Assignee: Wisconsin Alumni Research Foundation, Madison, WI (US)

(21) Appl. No.: 18/327,714

(22) Filed: Jun. 1, 2023

Related U.S. Application Data
(60) Provisional application No. 63/348,357, filed on Jun. 2, 2022.

(51) Int. Cl. G01N 1/40; G01N 1/38

(52) U.S. Cl. CPC G01N 1/4055; G01N 1/4044; G01N 1/38; G01N 2001/4061; G01N 2001/388

(57) ABSTRACT

Multi-omic analysis (analysis of proteins, lipids, and metabolites) is a powerful and increasingly utilized approach to gain insight into complex biological systems. One major hindrance with multi-omics, however, is the lengthy sample preparation process. Preparing samples for mass spectrometry (MS)-based multi-omics broadly involves extraction of metabolites and lipids with organic solvents, precipitation of proteins, and overnight digestion of proteins. The existing workflows are disparate and laborious, requiring multiple complex operation steps typically taking 1-2 days to perform. The present invention provides methods for preparing multi-omic samples that are faster and simpler than conventional methods, making it easier for a single lab or researcher to collect quality multi-omic data. A monophasic extraction solvent is used to efficiently extract biomolecules from a sample, including lipids and both polar and non-polar metabolites, and is paired with on-bead protein aggregation and rapid protein digestion.

### CROSS-REFERENCE TO RELATED APPLICATIONS

This application claims priority from U.S. Provisional Patent Application No. 63/348,357, filed Jun. 2, 2022, which is incorporated by reference herein to the extent that there is no inconsistency with the present disclosure.

### STATEMENT REGARDING FEDERALLY SPONSORED RESEARCH OR DEVELOPMENT

This invention was made with government support under GM108538 awarded by the National Institutes of Health. The government has certain rights in the invention.

### BACKGROUND OF THE INVENTION

Biological systems contain complex networks of diverse molecules that work together to modulate cellular processes. While many technologies interrogate a single biomolecule class, such as proteins, substantially more information and value can be gained from performing multi-omic studies, which profile multiple biomolecule classes simultaneously.

Integrated analysis of biomolecules was once a lofty goal; however, multi-omic investigations are now feasible and increasingly applied across biological disciplines,¹⁻³ in part due to improvements in mass spectrometry (MS) technologies. Evolving MS data acquisition strategies have increased the number of proteins, lipids, and metabolites surveyed;⁴⁻¹³ yet, the demand for faster and more efficient data collection persists. To meet this demand, simple, robust MS workflows must be developed that offer integrated analysis of multiple compound classes. While many recent advances have focused on improving integration of MS data acquisition and analysis strategies,¹⁴⁻¹⁸ other aspects of the MS multi-omic pipeline, such as sample preparation, are still in need of simplification and consolidation.

Sample preparation for published multi-omics studies involves either splitting samples into multiple aliquots for different -omes, or relying on extensive multi-step processes to isolate multiple compound classes.¹⁹⁻²⁸ These multi-step methods often require extracting metabolites and lipids with a biphasic organic solvent system, precipitating proteins, and digesting proteins with trypsin (FIG. 1). Most commonly, metabolites and lipids are extracted with the biphasic Matyash²⁹ (methyl tert-butyl ether [MTBE], methanol, water) or Folch/Bligh-Dyer^{30,31} (chloroform, methanol, water) solvent systems, which involve multiple organic solvents that are difficult to accurately pipette and require working in a fume hood.

And while robust and reproducible, these extraction methods require copious pipetting, vortexing, incubating, and centrifuging steps. Such steps are low throughput and susceptible to excessive sample loss. After phase separation, the lipid and metabolite layers are carefully aliquoted, and the protein pellet is then washed, dried, and resolubilized. Protein resolubilization in digestion buffer can be difficult and may require sonication or other facilitation methods. Subsequent overnight digestion of proteins adds 12-18 hours to the process, which is followed by desalting with solid phase extraction.

A more streamlined sample preparation would allow for simpler, faster, and more efficient ways to process lipids, metabolites, and proteins from a single sample and, when paired with an integrated acquisition method (e.g., multi-omic single-shot technology, MOST¹⁴), would allow a single lab or researcher to produce quality multi-omics data.

### SUMMARY OF THE INVENTION

The present invention provides a method for fast and efficient extraction and separation of biomolecules, including but not limited to lipids, peptides, nucleic acids, carbohydrates, metabolites, and combinations thereof, from a single sample for subsequent analysis. In particular, one aspect of the present invention provides a method for fast and efficient extraction of the lipidome, metabolome, and proteome of a cell for analysis.

One embodiment of the invention provides a method for extracting biomolecules from a sample comprising mixing the sample with an extraction solvent and a plurality of immobilizing beads. The extraction solvent is able to solubilize a first portion of biomolecules, which optionally comprises lipids, carbohydrates, metabolites, and combinations thereof. In an embodiment, the first portion of biomolecules comprises a mixture of lipids and metabolites from the sample. The plurality of immobilizing beads are able to bind and immobilize a second portion of biomolecules, which optionally comprises nucleic acids, proteins, polypeptides, and combinations thereof. In an embodiment, the second portion of biomolecules comprises a mixture of proteins and polypeptides from the sample. Optionally, mixing the sample with the extraction solvent and the plurality of immobilizing beads comprises incubating the sample with the extraction solvent and the plurality of immobilizing beads for an incubation time period between 5 minutes and 1 hour, preferably between 5 minutes and 30 minutes, more preferably between 5 minutes and 20 minutes.

The mixing step generates an extraction solution comprising the first portion of biomolecules and bound immobilizing beads, which are insoluble, attached to the second portion of biomolecules. The bound immobilizing beads attached to the second portion of biomolecules are separated from the extraction solution comprising the first portion of biomolecules. The first portion of biomolecules are then separated from the extraction solution, thereby generating at least a first set of extracted biomolecules, and the second portion of biomolecules are separated from the bound immobilizing beads, thereby generating at least a second set of extracted biomolecules.

The first and second portion of biomolecules, independently from one another, are optionally further separated into additional fractions, thereby generating a third set (optionally, a fourth set, a fifth set, a sixth set, etc.) of extracted biomolecules. For example, the first portion of biomolecules may be separated from the extraction solution to generate a set of lipid molecules and a separate set of metabolite molecules. Similarly, the second portion of biomolecules may be separated from the immobilizing beads to generate a set of polypeptides and a separate set of nucleic acids.

In an embodiment, the extracted biomolecules are then analyzed, which includes but is not limited to performing mass spectrometry analysis on at least the first set and second set of extracted biomolecules. In an embodiment, the mass spectrometry analysis comprises MS1 analysis, MS2 analysis, and combinations thereof. Preferably, the method is able to provide mass spectrometry analysis of the proteins and polypeptides of the sample, as well as mass spectrometry analysis of one or more of the lipids of the sample, the metabolites of the sample, the nucleic acids of the sample, and combinations thereof.

Conventional separation methods typically utilize a biphasic extraction solution. Preferably the extraction solution of the present invention is a monophasic solution. In an embodiment, the monophasic solution is able to solubilize at least 50% of both the lipids and metabolites of the sample, preferably at least 60% of both the lipids and metabolites of the sample, more preferably at least 75% of both the lipids and metabolites of the sample, more preferably at least 90% of both the lipids and metabolites of the sample.

The extraction solvent may be any solvent or combination of solvents able to solubilize the desired biomolecules from the sample for subsequent analysis. Optionally, the extraction solvent is a polar solvent or an amphiphilic solvent. In an embodiment, the extraction solvent comprises methanol, ethanol, propanol, butanol, water, acetone, ethyl acetate, dimethyl sulfoxide, hexane, methylene chloride, chloroform, diethyl ether, acetonitrile, or combinations thereof. Preferably, the extraction solvent comprises n-butanol and one or more co-solvents selected from the group consisting of methanol, ethanol, water, acetone, and acetonitrile. In an embodiment, the extraction solvent comprises n-butanol, preferably between 20% and 80% of n-butanol (by volume). Preferably, the extraction solvent comprises n-butanol and one or more co-solvents selected from the group consisting of methanol, ethanol, water, acetone, and acetonitrile. In an embodiment, the extraction solvent comprises, by volume, between 40%-70% n-butanol and 30%-60% water, and optionally one or more additional co-solvents. In a further embodiment, the extraction solvent comprises, by volume, between 50%-70% n-butanol, between 10%-30% acetonitrile, and between 10%-30% water. In a further embodiment, the extraction solvent comprises, by volume, between 55%-65% n-butanol, between 15%-25% acetonitrile, and between 15%-25% water.

In an embodiment, the immobilizing beads may be any type of bead or particle able to bind to proteins, polypeptides, nucleic acids, and desired negatively charged biomolecules as is known in the art (see as non-limiting examples, Hughes et al., Mol. Syst. Biol., 2014, 10:757; and Berensmeier, S., Appl. Microbiol. Biotechnol., 2006, 73(3): 495). In an embodiment, the immobilizing beads are magnetic or paramagnetic. In an embodiment, the immobilizing beads are silica beads or metal beads that are optionally coated. Optionally, the beads are functionalized, such as to contain antibodies, succinimide or carboxylate ligands, polymers, or carbohydrates. Preferably, the immobilizing beads are unmodified silica beads.

Optionally, the method further comprises digesting the proteins and polypeptides attached to the bound immobilizing beads. In an embodiment, the digestion step comprises mixing the bound immobilizing beads attached to the proteins, polypeptides, and combinations thereof, with a protein digestion enzyme, chemical agent, or combinations thereof. The protein digestion enzymes and chemical agents used herein may be any enzyme or chemical agent known in the art to digest or denature proteins and polypeptides for analysis, including but not limited to trypsin, pepsin, chymotrypsin, papain, calpain, serrapeptase, thermolysin, carboxypeptidase, acids, oxidizing or reducing agents, and combinations thereof. In an embodiment, the digestion step is performed for a time period between 15 minutes and 24 hours, between 2-24 hours, between 6-22 hours, between 8-20 hours, between 12-18 hours, between 10-16 hours, or between 8-12 hours. Preferably, the digestion step is a rapid digestion step performed for a time period between 20 minutes and 120 minutes, between 25 minutes and 90 minutes, between 30 minutes and 80 minutes, between 30 minutes and 60 minutes, between 35 minutes and 50 minutes, or between 35 minutes and 45 minutes. Preferably, the digestion step is performed at a temperature between 30° C. and 90° C., between 40° C. and 80° C., between 45° C. and 75° C., between 50° C. and 70° C., or between 55° C. and 65° C.

In an embodiment, the digestion step comprises mixing the bound immobilizing beads attached to the proteins, polypeptides, and combinations thereof, with a protein digestion enzyme, chemical agent, or combinations thereof for a digestion time period between 30 minutes and 60 minutes at a digestion temperature between 40° C. and 80° C., preferably for a digestion time period between 35 minutes and 45 minutes at a digestion temperature between 55° C. and 65° C. Optionally, the digestion step comprises using a protein digestion enzyme.

In an embodiment, the methods of the present invention (optionally including the digestion steps) are able to be completed within six hours, preferably within five hours, more preferably within four hours, more preferably within three hours, and more preferably within two hours. This is in contrast to conventional extraction and sample preparation methods which typically require 1-2 days.

Preferably, the sample comprises the proteome as well as the lipidome and/or metabolome of a cell, tissue, biological fluid (including but not limited to whole blood, plasma, saliva, cerebral spinal fluid, amniotic fluid, and synovial fluid), or combinations thereof. For example, the first portion of biomolecules (i.e., the portion solubilized by the extraction solvent) comprises the lipidome and metabolome of a cell, and the second portion of biomolecules (i.e., the portion attached to the immobilizing beads) comprises the proteome of the cell. In an embodiment, the sample is a whole cell lysate of one or more cells. In an embodiment, the sample is a solution or biological fluid, where the solution or biological fluid may or may not contain cells or components of lysed cells.

In an embodiment, the present invention provides a method for extracting biomolecules from a sample comprising the steps of: a) mixing the sample with an extraction solvent and a plurality of unmodified immobilizing beads, wherein the extraction solvent comprises, by volume, between 20% and 80% of n-butanol and is able to solubilize a first portion of biomolecules comprising lipids and metabolites, and wherein the plurality of unmodified immobilizing beads are able to bind and immobilize a second portion of biomolecules comprising proteins and polypeptides, thereby generating a monophasic extraction solution comprising the first portion of biomolecules and generating bound immobilizing beads attached to the second portion of biomolecules; b) separating the bound immobilizing beads attached to the second portion of biomolecules from the extraction solution comprising the first portion of biomolecules; c) mixing the bound immobilizing beads attached to the proteins and polypeptides with a protein digestion enzyme or chemical agent for a digestion time period preferably between 20 minutes and 120 minutes at a digestion temperature between 30° C. and 90° C.; and d) separating the first portion of biomolecules from the extraction solution, thereby generating at least a first set of extracted biomolecules, and separating the second portion of biomolecules from the bound immobilizing beads, thereby generating at least a second set of extracted biomolecules. In an embodiment, the digestion is performed for a time period between 30 minutes and 60 minutes at a digestion temperature between 40° C. and 80° C., more preferably for a digestion time period between 35 minutes and 45 minutes at a digestion temperature between 55° C. and 65° C. In an embodiment, steps a) through d) are performed within four hours or less, preferably within three hours or less, or preferably within two hours or less. In an embodiment, the method does not comprise any additional extraction steps performed on the sample, or additional separation steps.

In an embodiment, the present invention also provides a kit comprising the reagents and buffers described herein used to extract the desired biomolecules from the sample, including but not limited to the extraction solvent and immobilizing beads. For example, an embodiment of the invention provides a kit comprising an extraction solvent able to at least partially solubilize lipids, carbohydrates, biological metabolites, and combinations thereof, a plurality of immobilizing beads able to bind and immobilize polypeptides, and a digestion solution able to digest polypeptides, where the digestion solution comprises a protein digestion enzyme and/or a chemical agent. Optionally, kit further comprises one or more washing solutions, desalting solutions, buffers, and combinations thereof.

In an embodiment, the extraction solvent comprises methanol, ethanol, propanol, butanol, water, acetone, ethyl acetate, dimethyl sulfoxide, hexane, methylene chloride, chloroform, diethyl ether, acetonitrile, or combinations thereof. Preferably, the extraction solvent comprises 20-80% n-butanol and one or more co-solvents selected from the group consisting of methanol, ethanol, water, acetone, and acetonitrile. Preferably, the plurality of immobilizing beads comprise magnetic beads, paramagnetic beads, or silica beads. The protein digestion enzymes and chemical agents used herein may be any enzyme or chemical agent known in the art to digest or denature proteins and polypeptides for analysis, including but not limited to trypsin, pepsin, chymotrypsin, papain, calpain, serrapeptase, thermolysin, carboxypeptidase, acids, oxidizing or reducing agents, and combinations thereof. In an embodiment, the components of the kit are contained in separate containers from one another so that they may be added to one or more samples in separate steps. In an alternative embodiment, the extraction solvent and plurality of immobilizing beads are stored together and may be added to the one or more samples at the same time.

### BRIEF DESCRIPTION OF THE DRAWINGS

FIG. 1. General overview of conventional multi-omics sample preparation for MS analysis. Metabolites, lipids, and proteins are separated by an organic solvent system (e.g., MTBE/MeOH/H₂O), step (i), and the metabolite and lipid layers are removed for analysis, step (ii). The precipitated proteins are resolubilized and digested overnight with trypsin, step (iii). The resulting peptides are then desalted and prepared, step (iv). Metabolites, lipids, and peptides are analyzed on the respective instruments, step (v).

FIG. 2. Workflow for a BAMM preparation method in an embodiment of the present invention and comparison to published workflows. Panel a), to perform the BAMM workflow in an embodiment of the present invention, n-butanol-based monophasic solvent and magnetic beads are first added to a sample, step (1). After a brief vortex, the sample is incubated on ice for 5 minutes, step (2). Unbound metabolites and lipids are then removed, step (3), and protein is digested for 40 minutes at 60° C., step (4). Metabolites, lipids, and peptides are then prepared for analysis, steps (5-6). Panel b), comparison of the estimated number of steps necessary to prepare metabolites, lipids, and proteins with the BAMM method and various published methods. Panel c), comparison of the estimated number of hours it takes to perform the BAMM method and various published methods.

FIG. 3. Versatility and performance of BAMM Preparation method. The four left-hand columns (“Published Data”) show the number of proteins, lipids, and metabolites identified from conventional published multi-omic sample preparation workflows. All numbers were taken as reported in the results sections of the publications; however, the reported number of metabolites in Kang et al. was reduced to only those that matched the criteria for metabolite identifications used in this application. The five right-hand columns (“Data from BAMM Prep”) show the number of identified proteins, lipids, and metabolites from streamlined multi-omic sample preparation of this embodiment (numbers are the average of three replicates). The specific samples are as follows: A. thaliana (Kang et al.), S. cerevisiae (Stefely et al.), Calu-3 cells (Nakayasu et al.), mesenchymal stem cells (Coman et al.), and NIST 1950 plasma, S. cerevisiae, cultured murine adipocytes, C57BL/6J mouse brain, HEK293 (BAMM Prep).

FIG. 4. Example chromatograms for samples prepared with the BAMM method. Chromatograms for HEK293, panel a), and NIST 1950 plasma, panel b), analyses of peptides (top), lipids (middle), and metabolites (bottom), which were prepared with the BAMM method described in Example 2. The chromatograms are MS1, positive mode only.

FIG. 5. Determining the optimal type of magnetic bead for bead-based multi-omics sample preparation. Fold change in intensity of common metabolites, lipid classes (summed), and peptide GRAVY score between each bead type and no beads (MTBE extraction) from human plasma. Each bar represents the average of three technical replicates. The abbreviations used for the metabo-lites are as follows: cytidine 5′-diphosphocholine (CDP-choline), lysine (Lys), glycine (Gly), arginine (Arg), omithine (Om), 2-aminoadipic acid (2-AAA), aspartic acid (Asp), glu-tamic acid (Glu), glutathione (GSH), serine (Ser), nicotinamide (NAM), pantothenic acid (PA), and uridine monophosphate (UMP).

FIG. 6. Verifying relevant parameters for using magnetic beads in multi-omic sample preparation. Each bar represents the average of three technical replicates. Panel a), number of metabolites, lipids, and peptides from human plasma when varying the incubation period of the SeraSil-Mag 700 beads with the sample. Panel b), number of peptides from a plasma digest when varying the ratio of SeraSil-Mag 700 beads to protein amount.

FIG. 7. Development of a monophasic solvent system for metabolite and lipid extraction. Number of identified lipids, panel a), and metabolites, panel b), in human plasma for the monophasic n-butanol formulations compared to MAW, MTBE_{aq}, and MTBEₒᵣ controls. The percentage of n-butanol was balanced with H₂O (constant 20%) and ACN (0-80%). Sum peak areas of lipid features, panel c), and metabolite features, panel d), before and after dilution correction for MAW, MTBE, and 60% n-butanol/20% H₂O/20% ACN (“60% n-butanol”) extractions. Data are presented as mean±standard deviation. Class distributions for identified lipids, panel e), and metabolites, panel f), extracted with MAW, MTBE, and 60% n-butanol. Relative quantification in log₂(peak area) correlation plots, panel g), for lipids extracted with MTBEₒᵣ and 60% n-butanol. Panel h) shows metabolites extracted with MTBE_{aq} and 60% n-butanol; and panel i) metab-olites extracted with MAW and 60% n-butanol.

FIG. 8. Reducing overall time for proteomics sample preparation. Panel a), number of identified proteins from a mouse brain digest when using beads and performing both the 100% acetonitrile wash and 70% ethanol wash, only the 70% ethanol wash, only the 100% acetonitrile wash, and neither wash. Panel b), number of identified proteins from mouse brain digested overnight without beads, overnight with beads, and at 60° C. with beads. The beads used were Sera-Sil-Mag 700 nm beads.

FIG. 9. Optimizing and assessing the performance of accelerated on-bead protein digestion. Panel a), ratio of number of mouse brain peptides identified for each condition compared to a control overnight, no-bead digestion. The tested conditions were as follows: adding beads, varying temperature during digestion, adding movement during digestion, and varying length of digestion. Each bar represents the average of three technical replicates. Panel b), fold change in mouse brain peptide sum intensity between accelerated on-bead digestion and overnight no-bead digestion for missed cleavages, m/z, modifications, and peptide GRAVY score range. “All peptides” refers to summed intensities of all peptides identified by each condition, and “unique peptides” refers to summed intensities of only the peptides unique to each condition. Each bar represents the average of three technical replicates. The beads used were SeraSil-Mag 700.

FIG. 10. Comparison of extraction quality between monophasic and biphasic solvent systems. Panel a), comparison of sum peak areas of metabolite features in human plasma extractions for MeOH/ACN/H₂O (“MAW”), MTBE/MeOH/H₂O (“MTBE”), and the n-butanol formulations. Panel b), graphical illustration of the need for a correction factor between monophasic and biphasic extracts. Number of identified lipids, panel c), and metabolites, panel d), from human plasma when using the entire extract for MAW, MTBE, and n-butanol extractions. Sum peak areas of lipid, panel e), and metabolite, panel f), features in human plasma when using the entire extract for MAW, MTBE, and n-butanol extractions. Chromatograms (MS1, positive mode) comparing human plasma extractions with MAW, MTBE, and 60% n-butanol/20% H₂O/20% ACN (“60% n-butanol”) for lipids, panel g), and metabolites, panel h). Panel i), class distributions for identified metabolites extracted with MTBE aqueous phase and 60% n-butanol. Panel j), amino acid and lipid extraction recoveries for MAW, 60% n-butanol, MTBE aqueous layer, MTBE organic layer, and MTBE overall as determined from internal standards.

FIG. 11. Testing paramagnetic beads for proteomics. Monophasic extraction was combined with on-bead protein digestion (Hughes et al., J. Mol. Syst. Biol., 2014, 10 (10): 757; and Batth et al., Mol. Cell. Proteomics 2019, 18: 1027-1035). After adding in magnetic beads with sample and an extraction solvent, a short (˜10 min) incubation period would allow for protein aggregation on the beads, while metabolites and lipids remain unbound. The bead-bound proteins are then be digested. Panel a) shows different types of bead chemistries (carboxylate-coated hydrophobic (Hpb) and hydrophilic (Hpl) beads and unmodified silica 3 μm and 700 nm beads; mouse brain). Panel b) shows testing various ratios of bead to protein (yeast). Panel c) shows testing various incubation times for protein aggregation (plasma). Panel d) shows effects on washes after removing the metabolite and lipid supernatant (plasma).

FIG. 12. Analyzing effect of bead type on lipids. Intensities of lipid classes were summed to determine differences in the bead types. Overall, lipids did not appear to be affected by bead type or incubation times (data not shown).

FIG. 13. Analyzing the effect of beads on metabolites. Panel a) provides a heat map showing metabolites that varied the most in intensity between the different bead types. Panel b) provides a heat map showing differences in amino acid intensity when incubated with unmodified beads for varying amounts of time. Overall, the silica unmodified 700 nm beads produced the best results for proteins, metabolites, and lipids collectively.

### DETAILED DESCRIPTION OF THE INVENTION

### Overview

The analysis of proteins, lipids, and metabolites—multi-omics—is a powerful approach for gaining insights into complex biological networks and is increasingly applied across multiple disciplines (Krassowski et al., Frontiers in Genetics, 2020: 1598). Mass spectrometry (MS) is a prominent tool for multi-omic studies, offering robust and reproducible profiling of the proteome, lipidome, and metabolome. Preparing samples for MS-based multi-omic analysis broadly involves extraction of metabolites and lipids with a biphasic organic solvent system, precipitation of proteins, and overnight trypsin digestion (Stefely st al., Nat. Biotechnol. 2016, 34: 1191-1197). However, existing sample preparation for MS-based multi-omic analysis are laborious, disparate, and difficult to automate, requiring numerous pipetting, vortexing, and centrifugation steps along with protein resolubilization and solid phase extraction. Conventional sample preparation methods can further take 1-2 days to perform.

The present invention provides streamlined and efficient methods for preparing biomolecules, including but not limited to lipids, peptides, nucleic acids, carbohydrates, metabolites, and combinations thereof, from a single sample for mass spectrometry (MS) and other types of analysis.

In particular, the examples below describe a faster and simpler method to prepare samples for MS-based multi-omic analysis. In the specific examples described below, an n-butanol-based monophasic extraction solvent is used that efficiently extracts lipids as well as both polar and non-polar metabolites. The monophasic extraction is paired with paramagnetic bead technology for on-bead protein aggregation that requires only a short incubation time. Furthermore, the present methods may be used with a heated and rapid protein digestion step. After digestion, the protein solution is acidified, desalted, and dried down. The separate metabolite, lipid, and protein fractions are able to be resuspended for MS analysis.

Thus, the examples described below provide an improved multi-omic sample preparation method that enables faster (reduces preparation time by ˜94%) and simpler preparation (approximately ten steps vs. over twenty). The simplicity and time savings make it more amenable to a single lab technician preparing samples for same-day MS analysis. In addition, this process is more compatible with robotic automation and multi-well plate formats, which could significantly increase throughput.

Overall, this new strategy facilitates preparation of lipids, metabolites, and proteins in approximately three hours or less in some embodiments. This strategy eliminates several manual manipulations, centrifugations, tedious phase separation, and protein resolubilization steps. Despite the simplified steps, it was demonstrated that the performance of each part of the new workflow compares well to standard multi-omic workflows.

### EXAMPLES

Generally speaking, monophasic solvent extraction is combined with magnetic bead-peptide technology and an optional rapid digestion step, in order to expedite small molecule recovery and protein digestion. A sample comprising a mixture of lipids, peptides, metabolites and optionally nucleic acids is mixed with unmodified magnetic beads and a monophasic extraction solvent. A short incubation period facilitates protein aggregation on the beads, and the bead-bound proteins are separated from the monophasic solution containing unbound small molecules (such as lipids and metabolites). After small molecule removal, bead-bound proteins are enzymatically digested without the need for typical wash steps. The resulting peptides are then optionally desalted and purified, while the unbound molecules from the monophasic solution are similarly purified.

Compared to standard workflows, this new method reduced the total number of processing steps, eliminating several manual manipulations, centrifugations, tedious phase separation, and protein resolubilization steps. Despite the simplification of the process, biomolecule coverage and data quality were not compromised for any sample type. Prepared lipid, metabolite, and peptide samples are ready for MS analysis in approximately two to four hours, compared to approximately 1-2 days for standard workflows. Furthermore, compared to standard workflows, this method is more amenable to robotic automation and multi-well plate formats for increased throughput.

### Example 1—Conventional Multi-Omic Sample Preparation Workflow

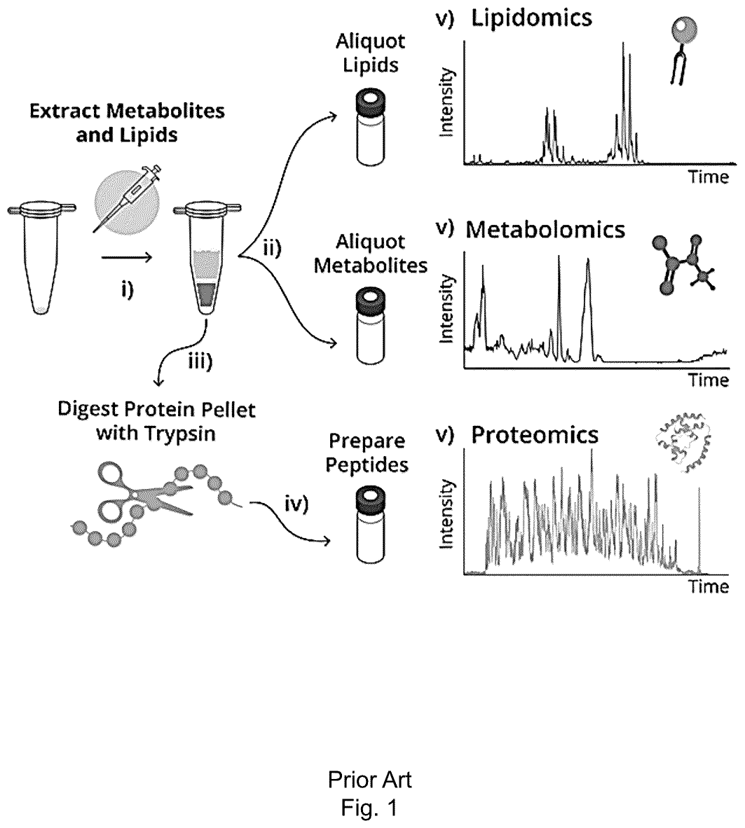

Published multi-omic sample preparation workflows vary widely in terms of their number of steps, solvent systems, digestion conditions, and overall throughput (see Kang et al.²⁶, Stefely et al.²⁴, Nakayasu et al.²³, and Coman et al.²²). FIG. 1 generally illustrates a conventional employed workflow and examples of the data it can generate.

One major drawback to such workflows is the standard biphasic solvent extraction. The two most common extraction systems are Matyash²⁹ (MTBE, methanol, water) and Folch/Bligh-Dyer³⁰⁻³¹ (chloroform, methanol, water). While robust, these extraction methods require multiple pipetting, vortexing, incubating, and centrifuging steps to achieve phase separation. After phase separation, the lipid and metabolite layers are removed, and the protein pellet is then washed, dried, and resolubilized.

Resolubilization of the proteins in digestion buffer can be difficult and may require sonication or other facilitation methods. Additionally, workflows relying on centrifugation to pellet the protein are not particularly amenable to limited amounts of starting material, as miniscule protein pellets are not easily visible. Subsequent digestion of proteins with Lys-C and trypsin typically adds 12-18 hours to the process, followed by desalting of peptides. In general, prepared lipids, metabolites, and peptides are ready for analysis after about a day or two. Overall, innovative strategies are needed to simplify this highly manual, tedious, and lengthy process to enable additional labs to prepare and analyze lipidomics, metabolomics, and proteomics data in-house.

### Example 2—Bead-Enabled Accelerated Monophasic Multi-Omics (BAMM)

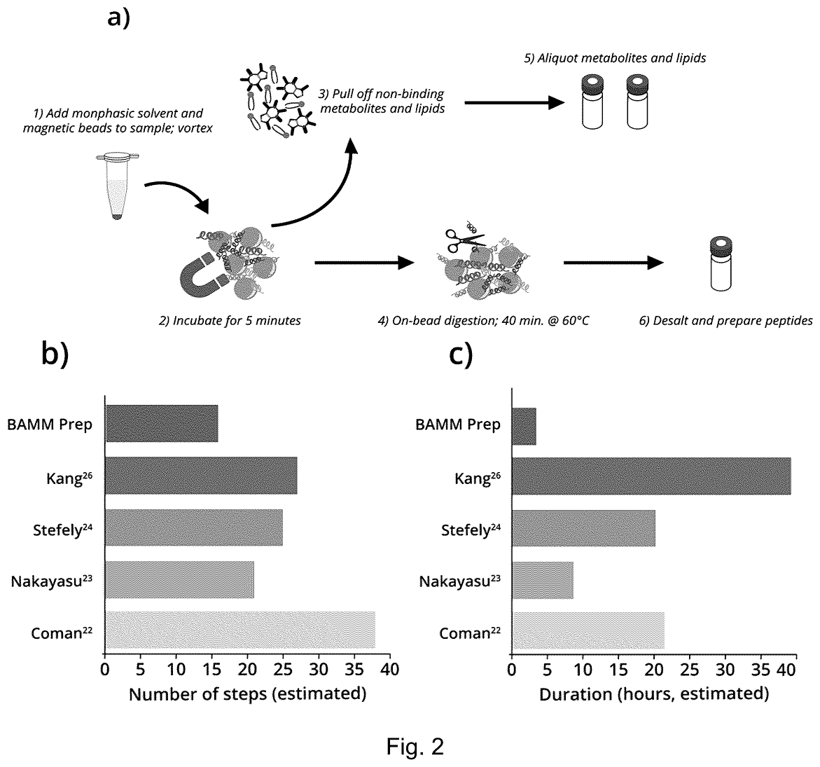

This example describes a faster, simpler method to prepare samples for multi-omic analysis that maintains similar biomolecular coverage and data quality as published methods.²⁴⁻²⁶ To simplify the preparation, a monophasic extraction system is used leveraging n-butanol's diverse miscibility³², with the goal of efficiently recovering both polar and non-polar metabolites. Next, a monophasic extraction is paired with paramagnetic bead technology for on-bead protein aggregation. In recent years, functionalized magnetic bead-based protocols have been introduced as an effective way to improve scalability, throughput, and flexibility for proteomics sample preparation, but they have not yet been tested for compatibility with metabolite and lipid extractions.³³⁻³⁷ Lastly, proteomic sample preparation time is reduced by implementing a heated, accelerated on-bead protein digestion with trypsin. As described further below, the strategy is able to eliminate several manual manipulations and reduces sample preparation time from 18+ hours to −3 hours. This particular embodiment is referred to as the Bead-enabled Accelerated Monophasic Multi-omics (BAMM) sample preparation for multi-omics analysis, and is generally illustrated in FIG. 2, panel a). Comparison of the BAMM method with published multi-omic sample preparation workflows, in terms of number of steps and the number of hours required to perform, are shown in FIG. 2, panel b).

Mouse Brain: All experiments were performed in accordance with the National Institutes of Health Guide for the Care and Use of Laboratory Animals and were approved by the Animal Care and Use Committee at the University of Wisconsin-Madison. Brains were harvested from C57BL/6J adult female mice after euthanasia and immediately frozen in liquid nitrogen. Tissues from several mice were combined and pulverized in liquid nitrogen; 15±2 mg of frozen pulverized brain was aliquoted into separate 1.5 mL microcentrifuge tubes and maintained at −80° C. until the time of extraction.

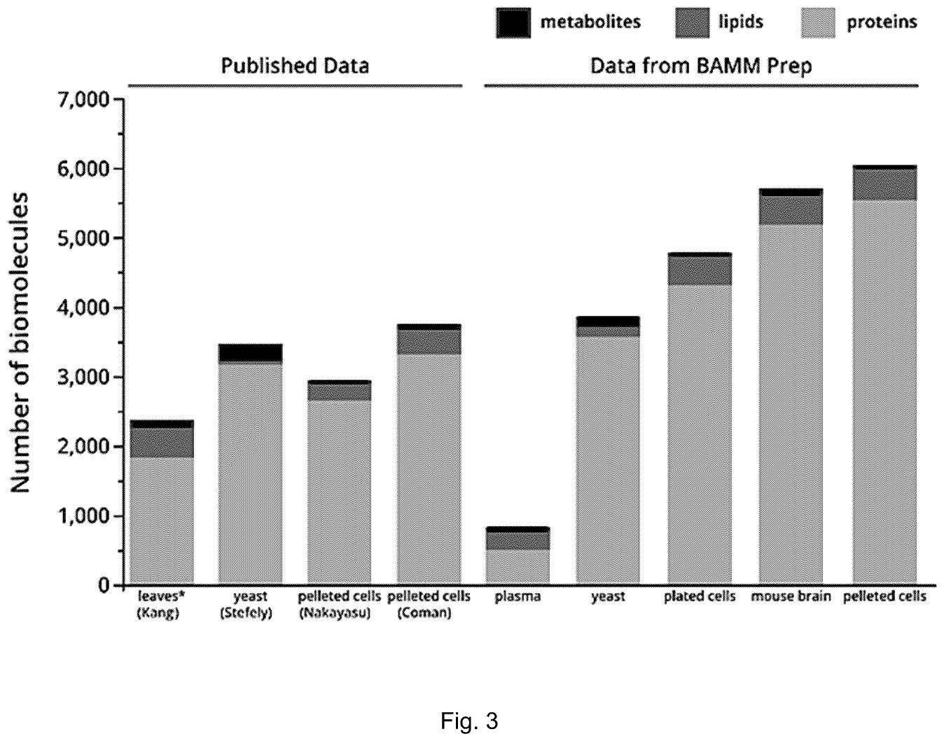

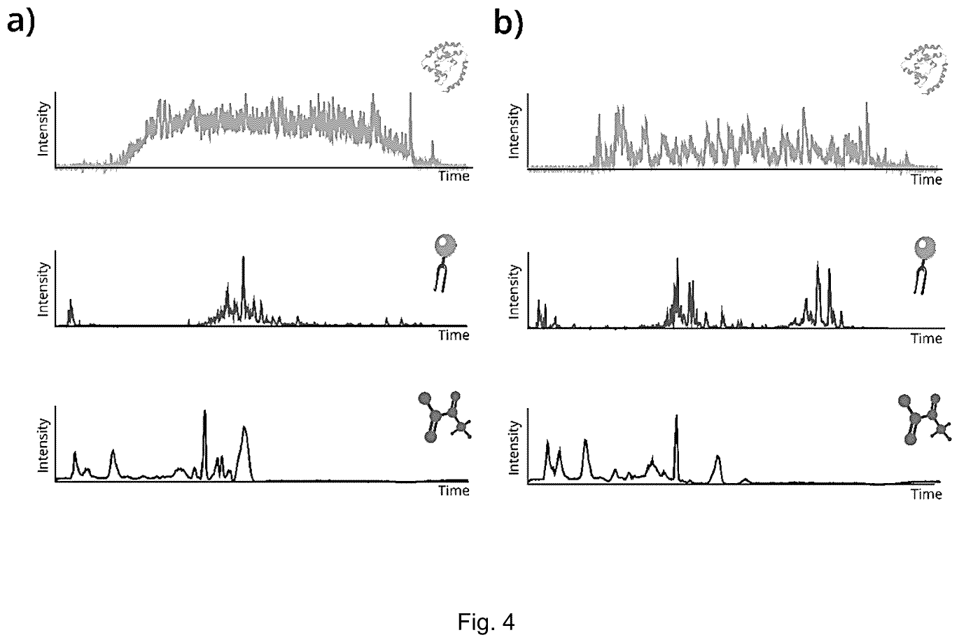

Human Plasma: Pooled, mixed-gender plasma sample was purchased from BioIVT (Human Plasma NaHep Lot #HMN378062) and used for all plasma experiments except for FIG. 3 and FIG. 4. The plasma sample in FIGS. 3 and 4 was from an aliquot of NIST Standard Reference Material 1950. For all experiments performed with plasma, 5 μL of plasma was extracted, unless otherwise noted.

Yeast: The Saccharomyces cerevisiae haploid W303 strain was grown in YPGD repository medium for 25 hours as previously described.¹⁴ Prior to extraction, the yeast cell pellets were flash-frozen in liquid nitrogen and maintained at −80° C. Each yeast pellet yielded about 500 μg of protein.

Adipocytes: Cultured adipocytes were prepared as previously described.³⁸ Briefly, differentiated adipocytes derived from murine mesenchymal precursors were trypsinized, seeded on cell culture microplates, and cultured for two days. Cell plates were then flash frozen in liquid nitrogen and maintained at −80° C. prior to extraction.

HEK293. In a standard tissue culture incubator, female HEK293 cells were cultured in DMEM (Thermo Fisher; high glucose with pyruvate and 4 mM glutamine) supplemented with 10% FBS at 37° C. and 5% CO₂. At approximately 70% confluency, cells were harvested by gentle washing with DPBS, scraping on ice in DPBS with 5 mM EDTA, and pelleting at 200×g for 4 minutes at 4° C. The resulting cell pellets were flash frozen in liquid nitrogen and stored at −80° C.

Metabolite and Lipid Standards: For recovery analysis, 5 μL of SPLASH Lipidomix internal standard mixture (Avanti Polar Lipids, Inc.) and 5 μL of Cell Free ¹³C, ¹⁵N Amino Acid Mixture (Sigma; diluted 1:100 from the stock) were added to samples for lipidomics and metabolomics analyses.

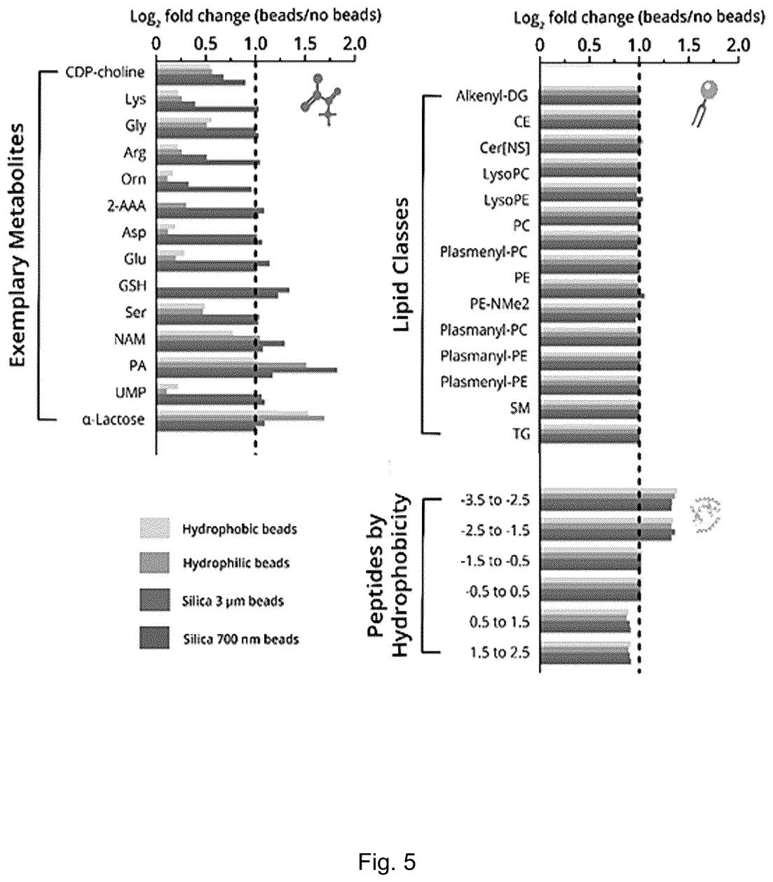

Preparation of Beads: Prior to performing experiments, Cytiva SeraSil-Mag 700 bead stock (or as specified, see FIG. 5) was taken in a quantity sufficient for a 10:1 bead-to-protein ratio. The bead stock was cleaned by washing once or twice with Nanopure water using a magnetic tube rack (Cell Signaling Technology). The beads were then reconstituted in Nanopure water. Prepared bead stocks in water were stored at 4° C. The amount of protein in each sample was either estimated based on prior knowledge or determined from a NanoDrop One Spectrophotometer (Thermo Scientific).

Biomolecule Extractions: For all metabolite/lipid extraction methods, samples were removed from −80° C. conditions and immediately placed on ice to thaw (plasma) or directly extracted from frozen material (cells and tissue). All extraction solvents were chilled and of liquid chromatography (LC)-MS grade.

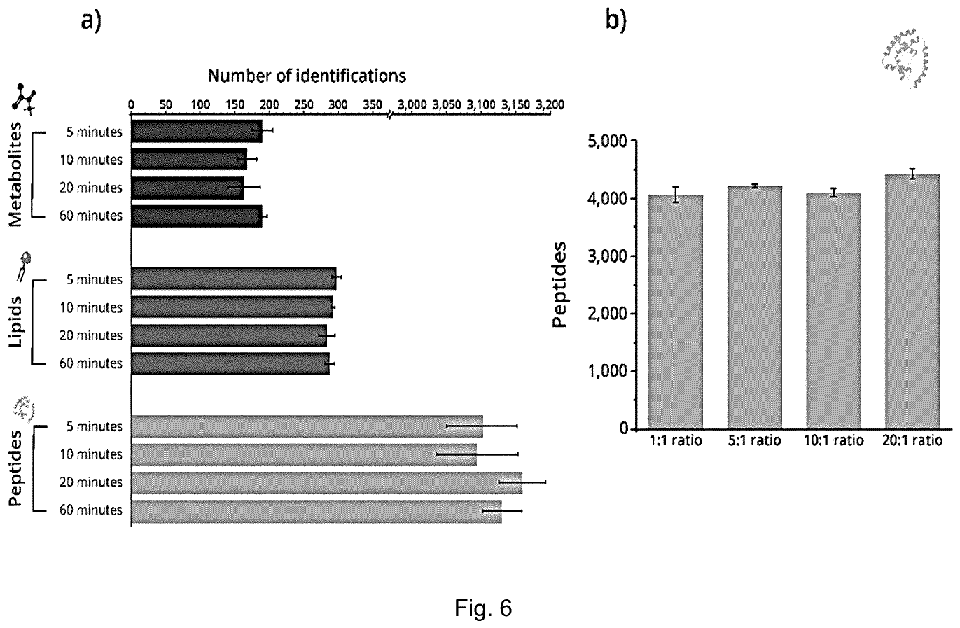

For monophasic extraction with beads, extraction solvent (60% n-butanol/20% ACN/20% H₂O) was added to each sample along with washed bead stock to achieve a 10:1 bead-to-protein ratio (or as specified, see FIG. 6) and a final water percentage of 20±1%. Cultured cells were detached using a plastic cell scraper³⁹; all other samples were vortexed for 10 seconds after the addition of solvents and beads. Cells and tissues were also sonicated in a chilled water bath (Qsonica) at 10° C. for a total of 5 minutes in increments of 20 seconds on/10 seconds off, with an amplitude of 30. Beyond facilitating cell lysis, sonication aided in the shearing of DNA⁴⁰, which resulted in improved proteomics results. Samples were then incubated for 5 minutes on ice (or as specified, see FIG. 6) and subsequently placed on a magnetic rack for 20 seconds. The resulting unbound supernatant containing metabolites and lipids was aliquoted into separate autosampler vials and dried with a SpeedVac Vacuum Concentrator (Thermo Scientific) for metabolomics and lipidomics analyses. Excess unbound supernatant was discarded.

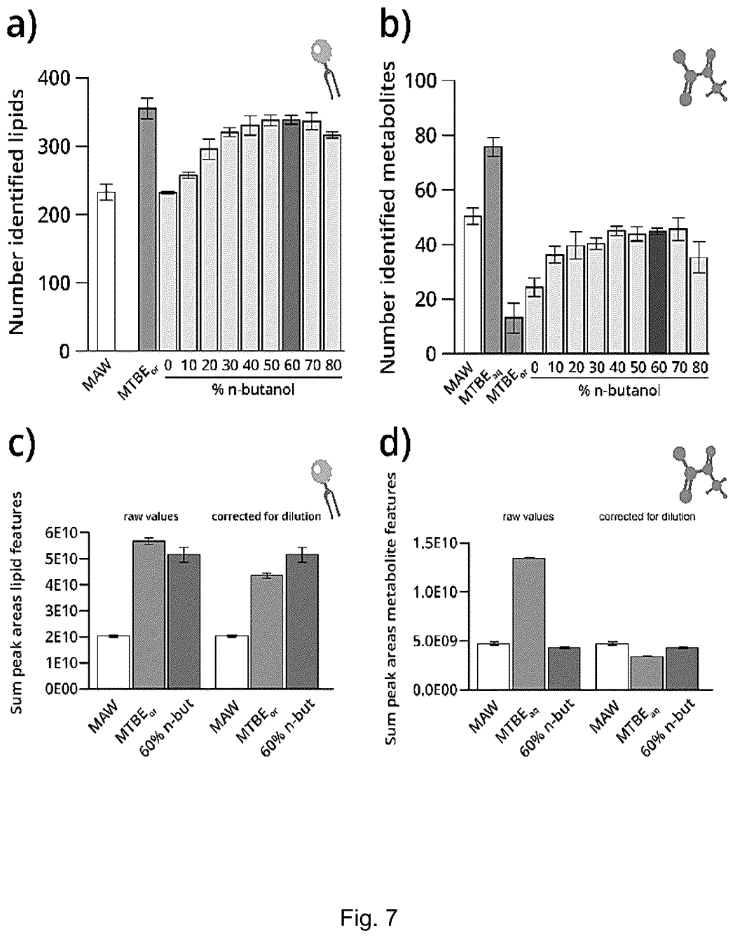

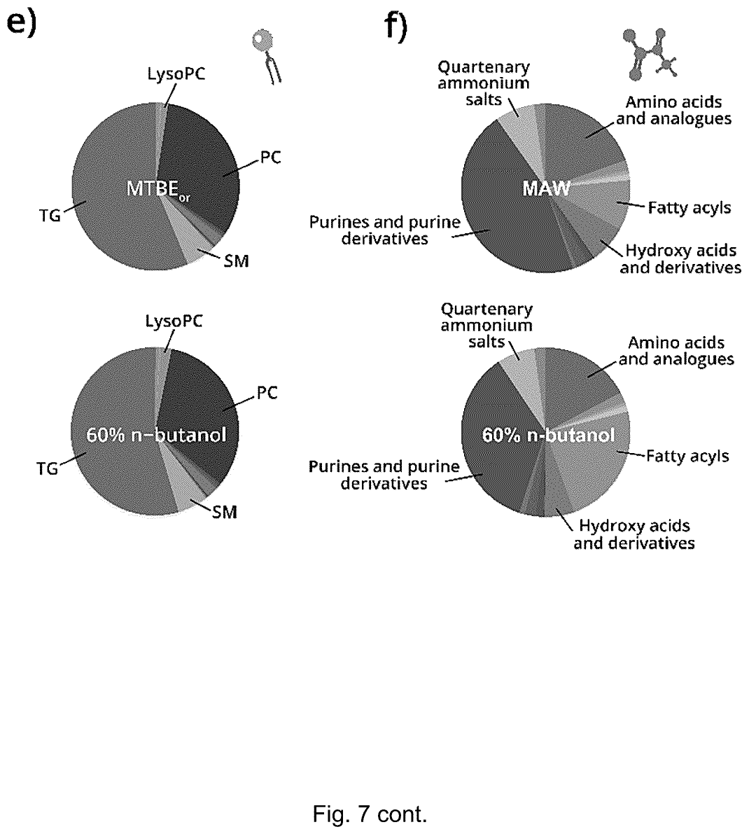

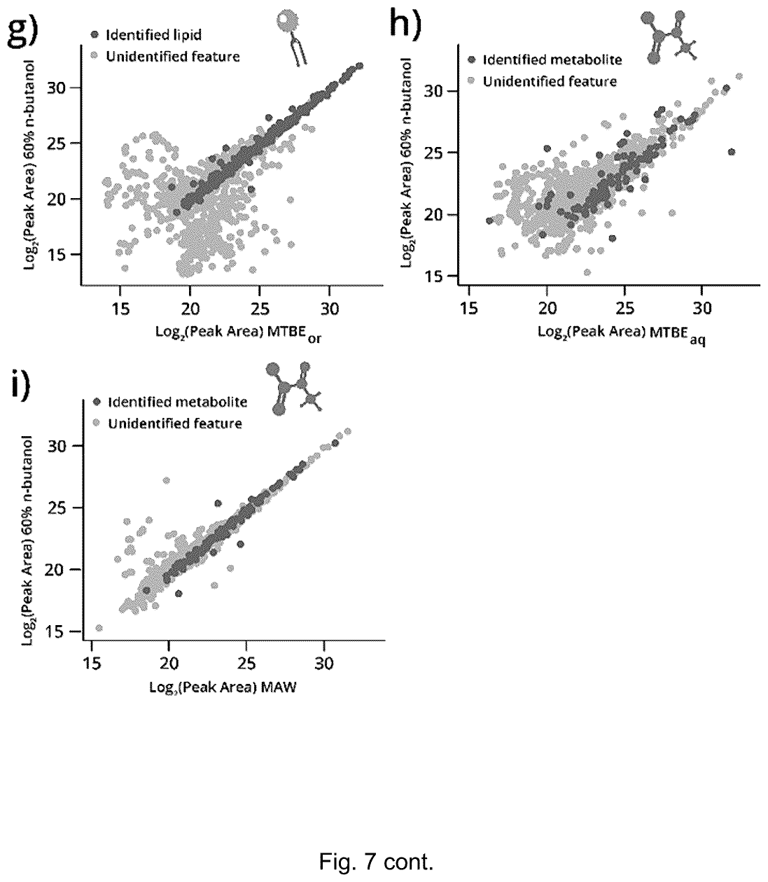

For extraction without beads, many n-butanol solvent systems were tested (see FIG. 7) and benchmarked against a common biphasic extraction system (Matyash²⁹; methanol/methyl tert-butyl ether/water (MeOH/MTBE/H₂O), 3:10:2.5 v/v/v) and a monophasic extraction system methanol/acetonitrile/water (MeOH/ACN/H₂O), 2:2:1 v/v/v). Solvents were added sequentially in the proportion described. Then, sample tubes were vortexed vigorously for 10 seconds, sonicated for 5 minutes at 14° C., incubated for 10 minutes at 4° C., and centrifuged at 14,000×g for 5 minutes at 4° C. The resulting supernatant layers were aliquoted into autosampler vials for metabolite and lipid analyses. For biphasic systems, the organic upper layer was used for lipid analysis and the aqueous bottom layer was used for metabolite analysis.

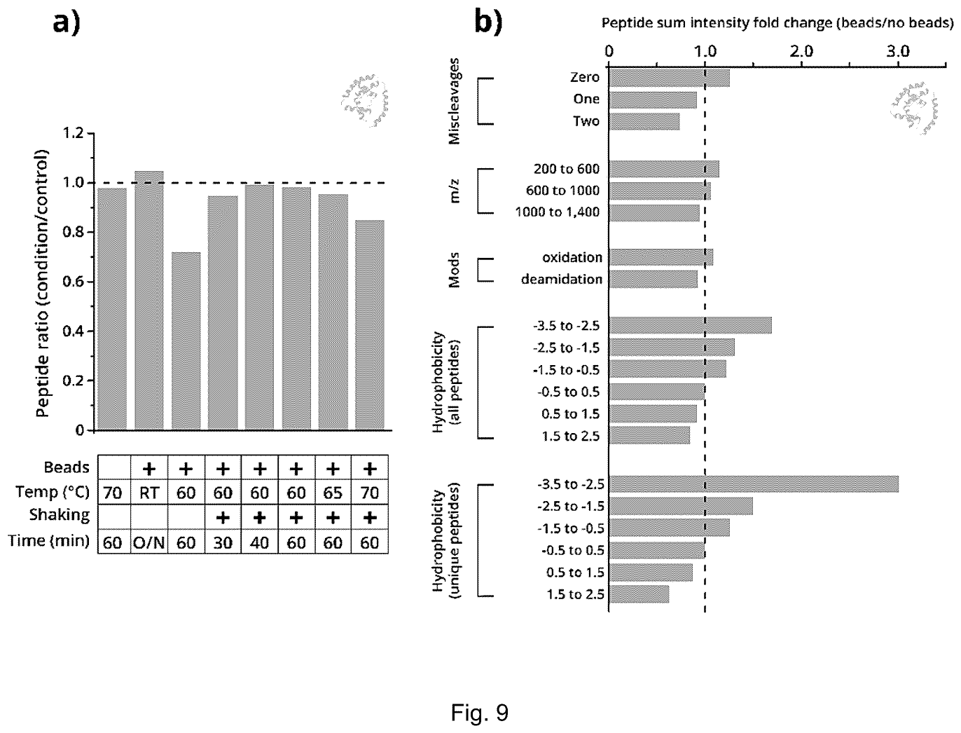

Accelerated Protein Digestion with Paramagnetic Beads: For on-bead accelerated protein digestion (see for example, FIGS. 3, 4, 8 and 9), after the metabolite and lipid supernatant was removed, the bead-protein mixture was reconstituted in digestion solution (Rapid Digestion Buffer [Promega] diluted to 75% from stock with spiked-in 5 mM TCEP, 20 mM CAA). Rapid Trypsin (Promega) was added in a 10:1 protein/enzyme ratio. The samples were incubated on a thermal mixer (Benchmark Scientific) at 1,000 rpm for 40 minutes at 60° C. (or as specified, see FIG. 9). Afterward, the tubes were placed on the magnetic rack, and the supernatant was recovered and acidified with trifluoroacetic acid to pH ˜2. The resulting peptides were desalted with Strata-×Polymeric Solid Phase Extraction cartridges (Phenomenex) and dried as described above.

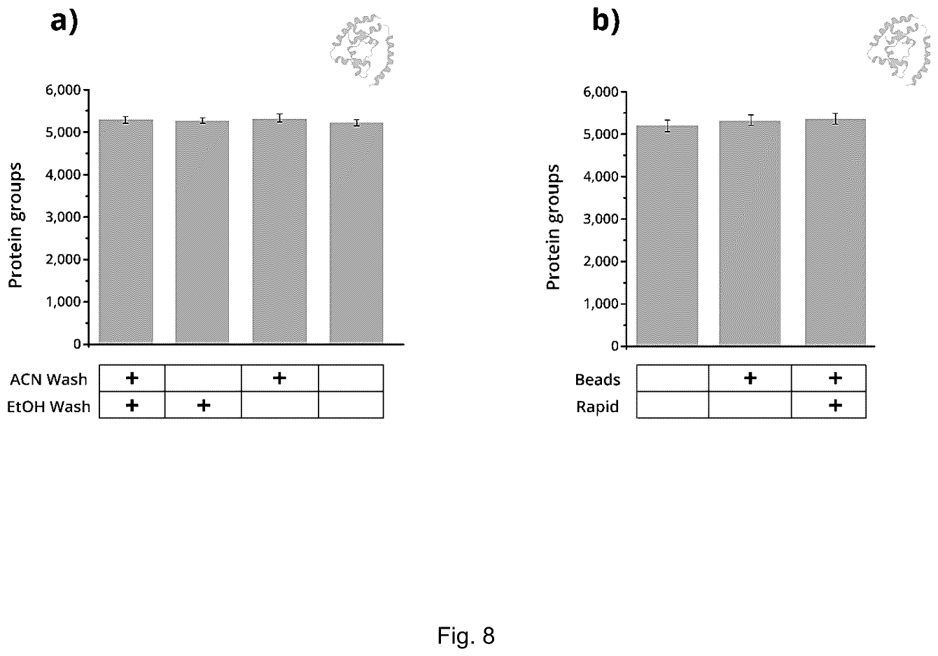

Overnight Protein Digestion with Paramagnetic Beads: When on-bead overnight protein digestion was performed (FIGS. 5-8), the metabolite and lipid supernatant was removed, and the bead-protein mixture was reconstituted in 50 mM Tris, 10 mM TCEP, 40 mM CAA. Trypsin (Promega) was then added in an estimated 50:1 protein/enzyme ratio. The samples were incubated overnight at room temperature on a rocker and acidified, desalted, and dried as described above. Note that originally, the bead-protein mixture was washed with 100% acetonitrile and 70% ethanol immediately following supernatant removal; however, these steps were removed in the final workflow because their elimination had minimal impact on results (see FIG. 8).

Protein Digestion without Magnetic Beads: For protein digestion without beads, the method was dependent on whether all -omes were analyzed (FIG. 5) or the tested variables were only relevant for proteomics (FIGS. 8 and 9). In the former case, supernatant was removed from the samples after biphasic extraction, and the protein pellets were washed with acetonitrile. In the latter case, the samples were suspended in lysis buffer (6 M guanidinium hydrochloride, 100 mM Tris) after thawing (plasma) or direct removal from frozen conditions (cells and tissues). Methanol was then added to each sample (90% v/v), and then samples were centrifuged for 5 minutes at 10,000×g. Proteins were resolubilized in digestion buffer (8 M urea, 10 mM TCEP, 40 mM CAA, 50 mM Tris) with 7.5 minutes of sonication. Before digestion, the samples were diluted to a final urea concentration of 1.5 M. Trypsin (Promega) was added in an estimated 50:1 ratio of protein/enzyme, and the samples were placed on a rocker for overnight incubation at room temperature. The resulting peptides were acidified, desalted, and dried as described above.

Lipidomics Data Acquisition and Analysis: For untargeted lipidomics LC-MS/MS analysis, dried supernatant aliquots were reconstituted in a 9:1 v/v methanol:toluene solution. To perform chromatographic separations, a Vanquish Split Sampler HT autosampler (Thermo Scientific) was used to inject 10 μL of reconstituted extract onto a Waters Acquity CSH C18 column (2.1×100 mm, 1.7 μm particle size) held at 50° C. throughout the analysis. Flow rate was maintained at 400 μL/min using a Vanquish Binary Pump (Thermo Scientific). The mobile phases consisted of 10 mM ammonium acetate in 70% ACN/30% H₂O (v/v) with 250 μL/L acetic acid (mobile phase A) and 10 mM ammonium acetate in 90% IPA/10% ACN (v/v) with the same additives (mobile phase B). A Q Exactive HF Orbitrap mass spectrometer (Thermo Scientific) coupled to a heated electrospray ionization (HESI-II) source was used for mass spectrometric detection. The source conditions were set as follows: HESI-II probe and capillary temperature, 350° C.; auxiliary gas temperature, 350° C.; sheath gas flow rate, 25 units; auxiliary gas flow rate, 15 units; sweep gas flow rate, 5 units; spray voltage, |3.5 kV| for both positive and negative ionization modes; and S-lens RF at 90 units. Data were acquired via polarity switching mode, acquiring full MS and MS/MS (Top2) spectra in both positive and negative ionization modes within the same injection. The acquisition parameters for full MS in both modes were set as follows: resolution of 30,000; automatic gain control (AGC) target of 1×10⁶; ion accumulation time (max IT) of 100 ms; and a scan range of 200-2000 m/z. MS/MS scans in both modes were then performed as follows: resolution of 30,000; AGC target of 1×10⁵; max IT of 50 ms; isolation window of 1.0 m/z; stepped normalized collision energy (NCE) at 20, 30, 40; and a dynamic exclusion of 30 seconds.

Lipidomics raw files were processed with Compound Discoverer 2.1 or higher (Thermo Fisher Scientific) and LipiDex.¹³

Metabolomics Data Acquisition and Analysis: For metabolomics LC-MS/MS analysis, dried supernatant aliquots were reconstituted in a 1:1 v/v acetonitrile:water solution. To perform chromatographic separations, a Vanquish Split Sampler HT autosampler (Thermo Scientific) was used to inject 2 μL of reconstituted extract onto a Millipore SeQuant ZIC-pHILIC column (2.1×100 mm, 5 μm particle size) held at 50° C. throughout the analysis. The flow rate was maintained at 150 μL/min using a Vanquish Binary Pump (Thermo Scientific). The mobile phases consisted of 10 mM ammonium acetate in 10% ACN/90% H₂O (v/v) with 0.1% ammonium hydroxide (mobile phase A) and 10 mM ammonium acetate in 95% ACN/5% H₂O (v/v) with 0.1% ammonium hydroxide (mobile phase B). A Q Exactive HF Orbitrap mass spectrometer (Thermo Scientific) coupled to a HESI-II source was used for mass spectrometric detection. The source conditions were set as follows: HESI II and capillary temperature, 350° C.; sheath gas flow rate, 40 units; auxiliary gas flow rate, 15 units; sweep gas flow rate, 1 unit; spray voltage, |3.0 kV| for both positive and negative ionization modes; and S-lens RF at 50 units. Data were acquired via polarity switching mode, acquiring full MS and MS/MS (Top10) spectra in both positive and negative ionization modes within the same injection. The acquisition parameters for full MS in both modes were set as follows: resolution of 60,000; AGC target of 1×10⁶; max IT of 100 ms; and a scan range of 70-900 m/z. MS/MS scans in both modes were then performed as follows: resolution of 45,000; AGC target of 1×10⁵; max IT of 100 ms; isolation window of 1.0 m/z; stepped NCE at 20, 30, 40; and a dynamic exclusion of 30 seconds.

Metabolomics raw files were processed with TraceFinder 3.3 (Thermo Fisher Scientific) using m/z and retention time tolerances for integration of specific metabolite features (see Table 1).

TABLE 1
Metabolite feature masses and retention times for integration.
“IS” refers to internal standard, “m/z”
refers to mass-to-charge, and “RT” time refers to retention time.

| Compound | Quan Mass (m/z) | RT (min) |
|---|---|---|
| Nicotinamide | 123.06 | 2.83 |
| O-Isovaleryl-L-carnitine | 246.17 | 6.00 |
| O-Butyryl-L-carnitine | 232.15 | 7.09 |
| Propionylcarnitine | 218.14 | 7.84 |
| Acetyl-L-carnitine | 204.12 | 8.72 |
| Nicotinic acid | 122.02 | 8.79 |
| L-Phenylalanine | 164.07 | 9.03 |
| L-Phenylalanine IS | 174.10 | 9.03 |
| DL-Leucine/Isoleucine | 130.09 | 9.20 |
| DL-Leucine_Isoleucine IS | 137.10 | 9.20 |
| Pantothenic acid | 218.10 | 9.30 |
| Xanthine | 151.03 | 9.30 |
| Xylitol to Arabitol | 151.06 | 9.85 |
| Indole-3-acrylic acid | 188.07 | 9.88 |
| Tryptophan | 205.10 | 9.88 |
| Tryptophan IS | 218.13 | 9.88 |
| DL-Proline | 116.07 | 10.16 |
| DL-Proline IS | 122.08 | 10.16 |
| L-Valine | 116.07 | 10.19 |
| L-Valine IS | 122.08 | 10.19 |
| DL-Carnitine | 162.11 | 10.56 |
| Guanosine | 282.08 | 10.80 |
| N-Acetylhistidine | 196.07 | 10.80 |
| Six-carbon sugar alcohol | 181.07 | 11.08 |
| L-Tyrosine | 180.07 | 11.15 |
| L-Tyrosine IS | 190.09 | 11.15 |
| S-Adenosylhomocysteine | 385.13 | 11.80 |
| L-Alanine | 88.04 | 11.98 |
| L-Alanine IS | 92.05 | 11.98 |
| N2-Acetyl-Lysine | 189.12 | 12.00 |
| Threonine | 118.05 | 12.12 |
| Threonine IS | 123.06 | 12.12 |
| DL-Glutamine | 147.08 | 12.71 |
| DL-Glutamine IS | 154.09 | 12.71 |
| L-Pyroglutamic acid | 130.05 | 12.71 |
| Glycine | 76.04 | 12.85 |
| Glycine IS | 79.04 | 12.85 |
| Lactose | 360.15 | 12.85 |
| Adenosine 5′-monophosphate | 348.07 | 12.90 |
| Gluconic acid | 195.05 | 13.00 |
| L-(+)-Citrulline | 176.10 | 13.00 |
| L-Serine | 104.04 | 13.10 |
| L-Serine IS | 108.04 | 13.10 |
| Cytidine 5′-diphosphocholine | 489.11 | 13.20 |
| L-Glutamic acid | 146.05 | 13.79 |
| L-Glutamic acid IS | 152.06 | 13.79 |
| D-Hexose 1-phosphate | 259.02 | 13.80 |
| L-2-Aminoadipic acid | 162.08 | 14.00 |
| L-Glutathione (reduced) | 306.08 | 14.13 |
| S-Adenosylmethionine | 399.14 | 14.20 |
| L-Aspartic acid | 132.03 | 14.22 |
| L-Aspartic acid IS | 137.04 | 14.22 |
| Uridine monophosphate (UMP) | 323.03 | 14.22 |
| L-Saccharopine | 277.14 | 14.40 |
| Adenosine diphosphate (ADP) | 426.02 | 14.50 |
| Cystathionine | 223.07 | 14.50 |
| Inosine-5′-monophosphate (IMP) | 347.04 | 14.50 |
| D-Sedoheptulose 7-phosphate | 289.03 | 14.80 |
| Guanosine 5′-monophosphate | 364.07 | 14.90 |
| Malic acid | 133.01 | 14.90 |
| Uridine†diphosphate†glucose | 565.05 | 14.90 |
| D-Erythrose-4-phosphate | 199.00 | 14.95 |
| L-Glutathione oxidized | 613.16 | 15.10 |
| Adenosine triphosphate (ATP) | 505.99 | 15.20 |
| Phosphoenolpyruvic acid | 168.99 | 16.00 |
| L(+)-Ornithine | 133.10 | 17.80 |
| Uridine 5′-diphosphoglucuronic acid | 579.03 | 17.80 |
| DL-Lysine | 147.11 | 17.92 |
| DL-Lysine IS | 155.13 | 17.92 |
| L-(+)-Arginine | 175.12 | 18.06 |
| L-(+)-Arginine IS | 185.13 | 18.06 |

Proteomics Data Acquisition and Analysis: For proteomics LC-MS/MS analysis, dried peptide samples were reconstituted in 0.2% formic acid in water. To perform chromatographic separations, a Dionex UltiMate WPS-3000RS autosampler (Thermo Fisher Scientific) was used to inject 1 μg of peptides onto a PicoFrit fused silica capillary column (New Objective) that was packed in-house⁵ to 30 cm with 1.7 μm, 130 Å pore size C18 BEH particles (75×360 μm). The column was held at 50° C. with an in-house built heater. The flow rate was maintained at 300 nVmin. The mobile phases consisted of 0.2% formic acid in water (mobile phase A) and 0.2% formic acid in 80% ACN/20% H₂O (v/v, mobile phase B). The LC was coupled to an Orbitrap Eclipse (Thermo Fisher Scientific) via a nano ESI source for mass spectrometric detection. The transfer capillary temperature was set to 275° C., and the positive spray voltage was 2.5 kV. A one-second cycle time was used to acquire full MS and MS/MS scans in “Top Speed” mode. The acquisition parameters for full MS were set as follows: resolution of 240,000 in the Orbitrap, AGC target of 1×10⁶; max IT of 50 ms, and a scan range of 300-1350 m/z. The APD algorithm was toggled on.⁷ MS/MS scans were performed as follows: turbo scanning mode in the ion trap, AGC target of 3×10, max IT of 14 ms, isolation window of 0.5 m/z, NCE of 25%, and dynamic exclusion of 10 s. Charge states 2-5 were included, and the default charge state was 2.

MaxQuant⁴ (version 1.5.2.8) was used to search all proteomics raw files. The appropriate references database was downloaded from UniProt (human, mouse, or yeast; canonical and isoforms). Searches were performed using the Andromeda⁴² search algorithm and label-free quantification.⁴³ Default parameters were used. Match between runs was not applied unless noted.

Dilution corrections: To compare extraction solvents, metabolite and lipid extractions were corrected for differences in dilutions between monophasic and biphasic extractions. Correction factors were calculated as the sum of the measured volumes (aqueous phase+organic phase) divided by the volume of the phase used for analysis, and normalized total volumes across all samples. Actual biphasic volumes were estimated using a glass Hamilton pipettor

Monophasic Solvent System for Lipid and Polar Metabolite Extraction: Initial efforts toward a simplified multi-omics workflow involved optimizing a monophasic solvent system for lipid and metabolite extraction. In standard biphasic solvent systems, lipids partition into a strongly lipophilic solvent (e.g. chloroform,³⁰⁻³¹ MTBE²⁹), while polar metabolites partition into the aqueous phase. The biphasic systems successfully extract a wide range of compound classes. In contrast, current monophasic extraction methods, although simpler, tend to preferentially extract either lipophilic or polar metabolites.^{39,44-48} With the goal of developing a monophasic solvent system that recovers both lipids and small molecules with high efficiency, aqueous n-butanol mixtures were explored, as they have been described³² as containing properties compatible with both polar and non-polar compounds.

First, a range of n-butanol formulations (0-80% n-butanol) were tested for suitability to extract lipids and polar metabolites. The proportion of water was maintained at 20% (v/v), and acetonitrile was used to balance the proportion of n-butanol. From 0-60% n-butanol, the solvents remained miscible, yielding the desired monophasic extraction solvent. However, at 70% and 80% n-butanol, slight and moderate phase separation was induced, respectively. Using 500 μL of each solvent mixture, 10 μL of human plasma were extracted and analyzed with equal portions of extract (100 μL) by LC-MS/MS for lipids and metabolites. For the phase-separated n-butanol extracts, the upper layer was used for both metabolite and lipid analyses. All extracts were dried by vacuum centrifugation and resuspended in the same solvent for analysis. The n-butanol formulations were compared to a common metabolomics monophasic solvent⁴⁸ (2:2:1 MeOH:ACN:H₂O, “MAW”) and the traditional biphasic Matyash solvent system²⁹ (10:3:2.5 MTBE:MeOH:H₂O, “MTBE”), using the same ratio of plasma to solvent.

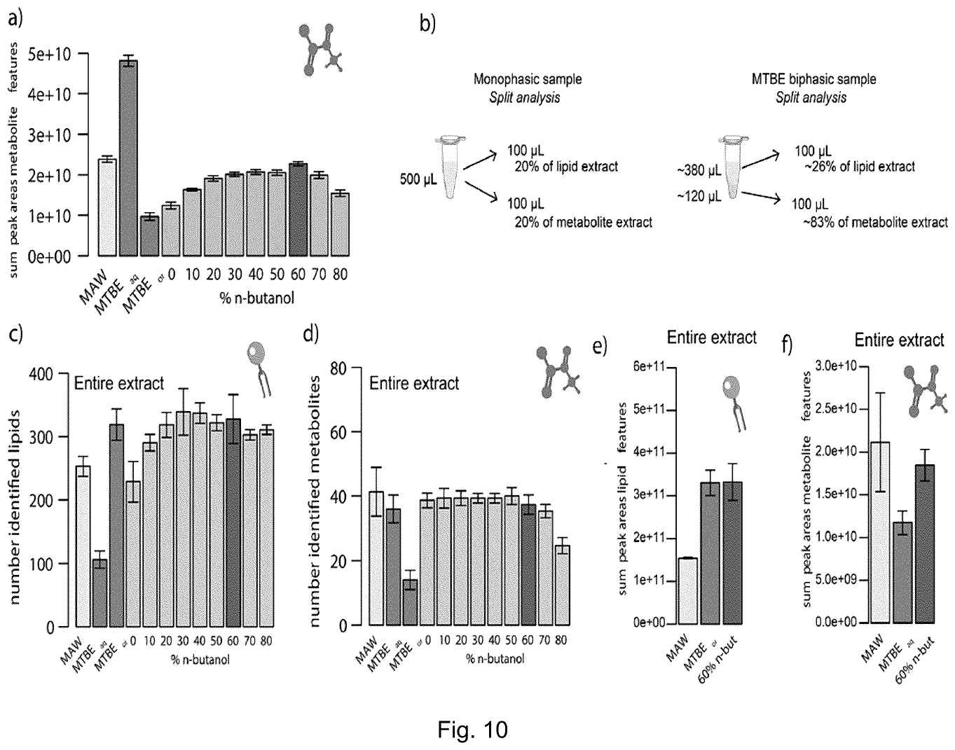

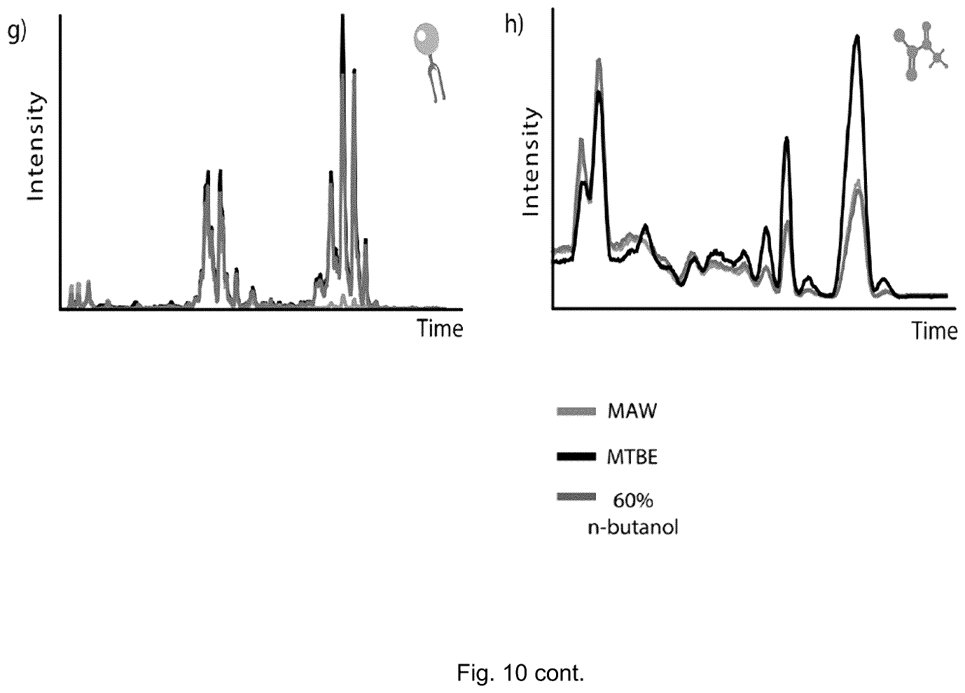

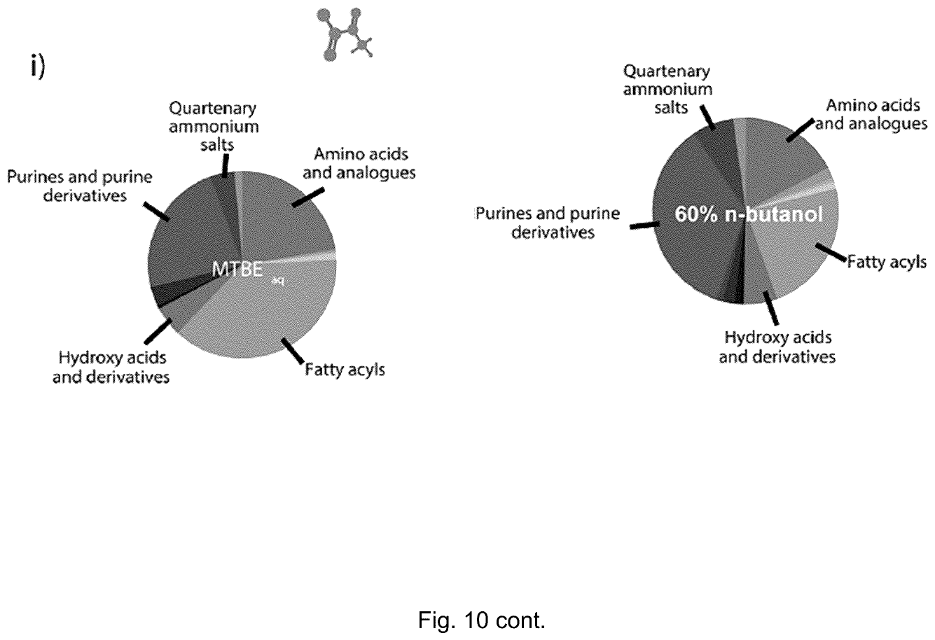

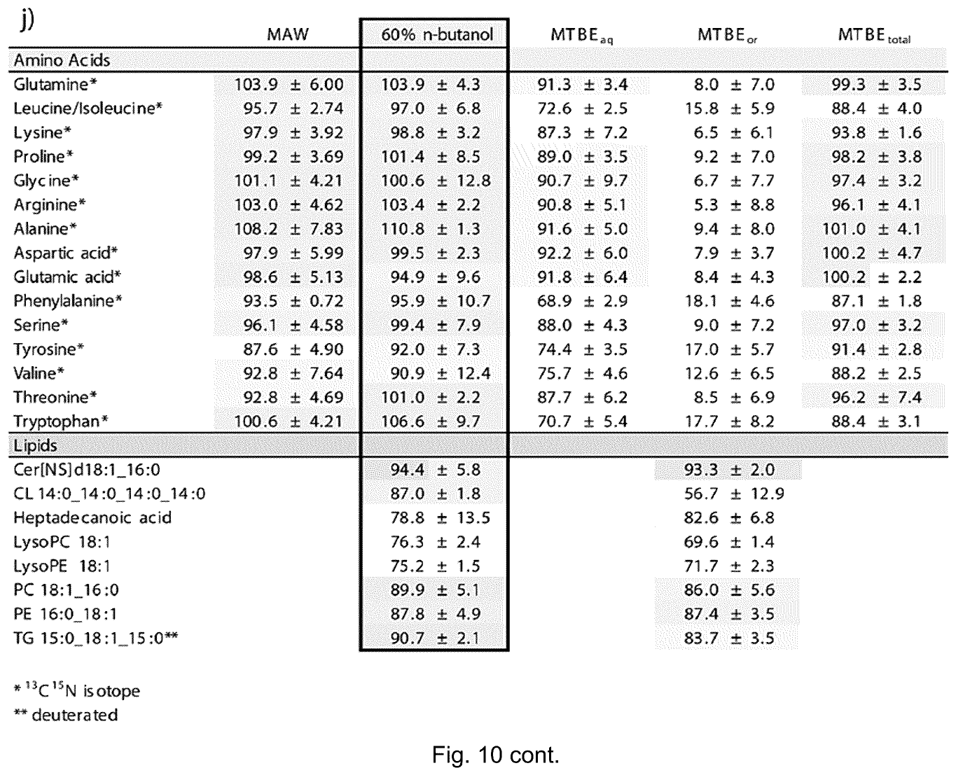

To evaluate the extraction solvents, the number of lipids and metabolites identified were first assessed (FIG. 7, panels a-b)). The n-butanol formulation that yielded the most lipid identifications was the 60% n-butanol/20% ACN/20% H₂O. The 40-70% n-butanol formulations yielded similar numbers of metabolite identifications, but the 60% n-butanol extraction was higher than the others in sum metabolite intensity (FIG. 10, panel a). Therefore, 60% n-butanol/20% ACN/20% H₂O was the best-performing monophasic solvent system when considering both lipids and metabolites. In comparison to the MAW control, the 60% n-butanol yielded markedly more lipids and a similar number of metabolites. The MTBE control recovered more identifications for both lipids and metabolites; however, this difference was due to MTBE being more highly concentrated (FIG. 10, panel b). When correcting for concentration differences, the 60% n-butanol extraction system compared favorably with the MTBE extraction for both lipids and metabolites (FIG. 7, panels c-d)). Likewise, if the entire monophasic extract is used for either lipid analysis or metabolite analysis, the resulting number of identified metabolites and lipids is not different between MTBE and 60% n-butanol extractions (FIG. 10, panels c-d), and the sum feature intensities for these analyses closely mirror the dilution corrected values in FIG. 7, panels c-d) (see FIG. 10, panels e-f). For comparison, overlaid chromatograms of 60% n-butanol and both controls are shown in FIG. 10, panels g-h).

After determining that 60% n-butanol was optimal, class distributions of extracted lipids and metabolites were assessed (FIG. 7, panels e-f)). The MTBE organic phase (MTBEₒᵣ) and 60% n-butanol recovered the various lipid classes in similar proportions. Notably, the 60% n-butanol successfully extracted both hydrophilic lipids, such as lyso-phospholipids, and hydrophobic lipids, such as triglyceride lipid species. For metabolomics, 60% n-butanol was compared to MAW and the MTBE aqueous phase (MTBE_{aq}). The distribution of metabolite classes recovered by MAW and 60% n-butanol were similar, but MAW recovered a larger percentage of purines and purine derivatives, while 60% n-butanol recovered more fatty acyls. Comparing 60% n-butanol to MTBES_{aq} (FIG. 10, panel i)), greater differences in relative compounds extracted were observed. It is believed this is in part due to the difference in metabolite recoveries between the two solvent phases (FIG. 10, panel j)). Calculating extraction recoveries revealed not only high overall recovery of both lipid (mean 85±6%) and amino acid (mean 99±8%) internal standards with 60% n-butanol, but also that the mean recovery by 60% n-butanol was higher than MTBE_{aq} for all amino acids. Hydrophobic amino acids specifically are disadvantageously split between the aqueous and organic layers in the biphasic system. For example, recovery of tryptophan is 51% higher with 60% n-butanol compared to MTBE_{aq}.

Finally, quantitative correlation (relative abundance) was examined between 60% n-butanol and MTBEₒᵣ for lipids (FIG. 7, panel g)), 60% n-butanol and MTBE_{aq} for metabolites (FIG. 7, panel h)), and 60% n-butanol and MAW for metabolites (FIG. 7, panel i)). Strong correlations were observed for all, indicating that 60% n-butanol recovers lipid and metabolite species in similar proportions as the respective controls. Overall, the 60% n-butanol/20% acetonitrile/20% water monophasic system proved to efficiently recover both lipids and polar metabolites. However, compared to traditional biphasic MTBE systems, the monophasic system is simpler and does not require phase separation or additional incubation and pipetting steps.

Magnetic Beads to Facilitate Integrated Sample Preparation: Expanding on the simplicity of the monophasic solvent for metabolomics and lipidomics sample preparation, this extraction was integrated with paramagnetic bead technology to expedite proteomics preparation. The use of magnetic beads for proteomics (termed the SP3 approach³³⁻³⁶) was introduced in recent years as a universal sample preparation platform. The SP3 protocol uses carboxylate-coated hydrophilic magnetic beads in the presence of high organic solvent to induce protein-bead aggregation. Once proteins are immobilized on the surface of the beads, they can be rinsed of contaminants (e.g. chaotropes, detergents), released, and digested. Here, a modified SP3 approach was envisioned that would be amenable to multi-omics. First, magnetic beads were added to monophasic solvent and sample, and proteins were allowed to aggregate around the beads during a short incubation period. Unbound metabolites and lipids were removed for further analysis, and bead-bound proteins were rinsed, digested, and desalted. Overall, the goal was to eliminate centrifugation and protein resolubilization by combining a bead-based protocol with the monophasic solvent extraction. However, because this SP3 method has not been demonstrated for compatibility with metabolite or lipid analyses, it was hypothesized that different functional groups on the beads may influence extractions of metabolites and/or lipids.

Four different types of magnetic beads were obtained to test with multi-omic extractions: 1 μm hydrophilic carboxylate functionalized beads (Cytiva), 1 μm hydrophobic carboxylate functionalized beads (Cytiva), 3 μm unmodified silica beads (G-Biosciences), and 700 nm unmodified silica beads (Cytiva). Even though the SP3 protocol is typically performed with carboxylate functionalized beads, it has been shown³³ that proteins are not influenced by specific bead properties and thus aggregate on any available surface upon conditions known to induce aggregation. To examine the performance of the four bead types, metabolites, lipids, and proteins were extracted from plasma with each bead and without beads. The log₂ fold changes in intensity were compared between each bead type and the no-bead control for common metabolites, lipid classes, and peptide GRAVY (grand average of hydropathicity index; a measure of hydrophobicity) score range⁴⁹ (FIG. 5). Lipid and peptide identifications were thoroughly unaffected by the type of bead used, as their fold changes for each bead type displayed only minimal fluctuations. Metabolites, on the other hand, were quite affected by the type of bead used. In particular, recovery of the most polar metabolites was hindered with the functionalized beads. Recovery improved somewhat with the 3 μm unmodified beads, but the best-performing type was the 700 nm unmodified beads. Extracting metabolites in the presence of the 700 nm unmodified beads led to similar recovery as without beads for all metabolites. Similar results were observed when these experiments were performed with mouse brain for lipids, metabolites, and peptides (data not shown).

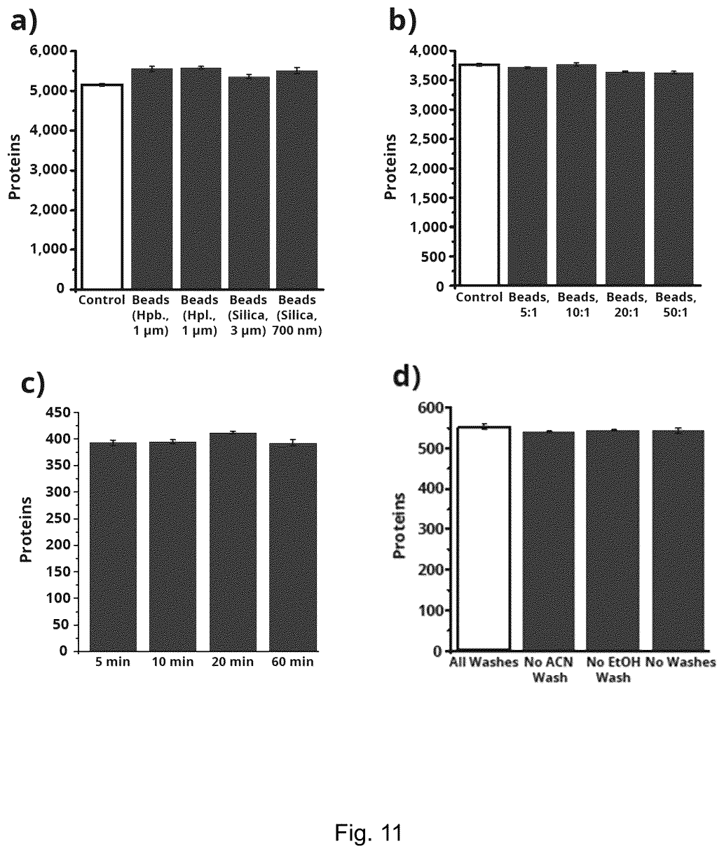

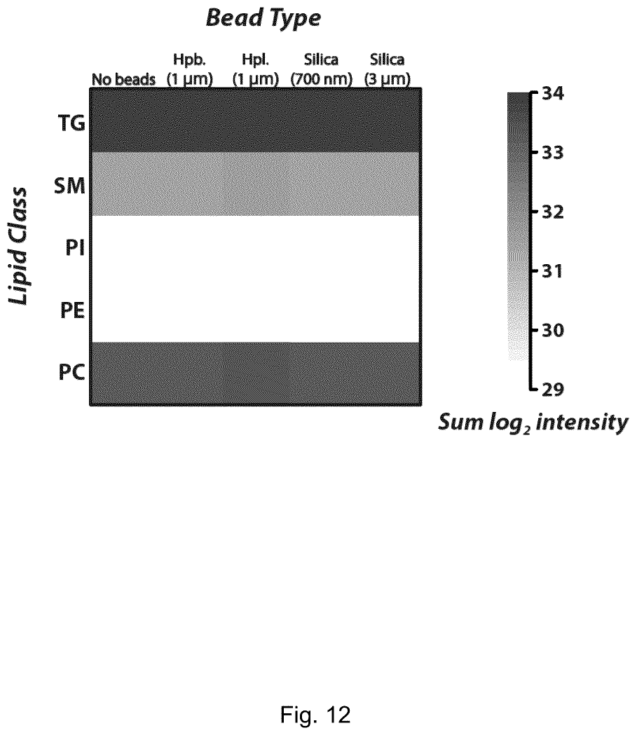

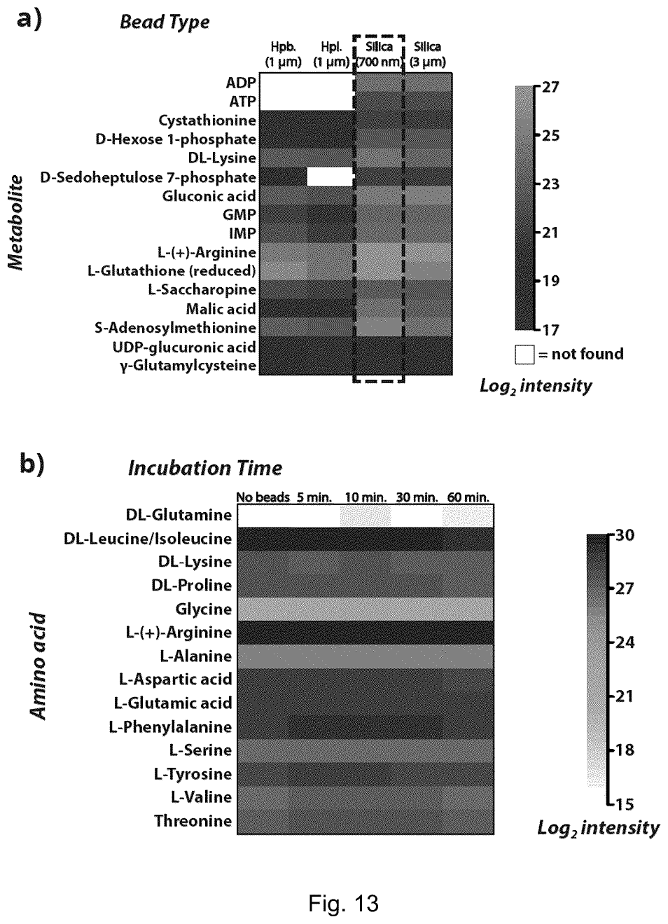

The reduction in the recovery of certain metabolites when using functionalized beads is likely due to inadvertent capture of those metabolites by the bead surface. Interestingly, the 700 nm nonfunctionalized beads avoid this problem, but the 3 μm nonfunctionalized beads avoid it only partially. Some metabolites likely still have a partial interaction with the silica surface⁵⁰ of the 3 μm beads, and size may potentially play a role. Regardless, the 700 nm unmodified beads were clearly optimal over the other bead types for metabolites; therefore, these beads were chosen for subsequent experiments and final multi-omics workflow. After establishing the bead type, it was verified that bead surface interaction with biomolecules was not time-dependent, as little to no difference in metabolite, lipid, and peptide recovery from plasma were seen when varying the incubation period of the beads with the sample from 5 to 60 minutes (FIG. 6, panel a)). It was also confirmed that the ratio of bead-to-protein had minimal effect on plasma peptide yields; bead-to-protein ratios of 1:1, 5:1, 10:1, and 20:1 yielded similar results (FIG. 6, panel b); identical results in yeast not shown). SP3 protocols recommend a 10:1 bead-to-protein ratio³⁶; therefore, this ratio was used for the final workflow. Additional experiments showing the effects of bead type, bead-to-protein ratios, and incubation times are shown in FIGS. 11-13.

These experiments confirmed that the SP3 method for proteomics can be expanded for multi-omics, preferably with a nonfunctionalized bead. A bead-based multi-omics workflow not only consolidates the preparation but also eliminates the need for centrifugation and protein resolubilization, as aggregated proteins are digested directly on-bead.

Reducing Overall Preparation Time By Accelerating Proteomics Preparation: As a final simplification to the multi-omics workflow, opportunities to reduce the overall time taken for proteomics sample preparation were explored, which is the lengthiest portion of the process. First, it was attempted to remove any unnecessary steps, such as wash steps between removal of the metabolite and lipid supernatant and addition of digestion buffer. In current SP3 protocols, it is typical to perform 2-3 wash steps (often with acetonitrile and/or ethanol) before digestion.^{38,37} The intent of the washes is to remove detergents and contaminants; however, detergents are largely incompatible with MS-based metabolomics and lipidomics, and therefore it was reasoned that wash steps are not as necessary with multi-omics samples. The experiments in FIGS. 5 and 6 were performed with a 100% acetonitrile wash followed by a 70% ethanol wash. However, as shown in FIG. 8, panel a), removing both wash steps for a mouse brain digest resulted in only 3% fewer protein identifications (identical result in plasma, not shown). Therefore, the washes did not appear to be necessary and were removed for the final workflow.

Second, enabling same-day analysis of all three -omes was explored. Metabolite and lipid samples can typically be prepared and analyzed on the instrument within the same day; however, the typical overnight digestion of proteins stalls peptide analysis until at least the following day. To expedite digestion, it was attempted to combine Promega Rapid Trypsin with on-bead protein digestion. The Rapid Trypsin platform reduces protein digestion time to one hour or less by heating samples up to 70° C., which requires a specialized non-urea buffer and thermostable trypsin.⁵¹ The Rapid Trypsin protocol was optimized to be compatible with magnetic beads, which required the sample to be shaken throughout the digestion period (˜35% increase vs. not shaken) and the temperature to be lowered to 60° C. from 70° C. (FIG. 9, panel a)). With these modifications, the digestion step was reduced from 12+ hours to 40 minutes without loss of depth or quality as compared to both a no-bead and bead overnight digestion (FIG. 8, panel b), and FIG. 9, panel b)). Despite this reduced duration, semi-tryptic cleavage rates and sequence coverage were comparable for both the rapid bead and overnight no-bead workflows (<1% and ˜30%, respectively). About 10-20% fewer missed cleavages were observed with the rapid bead workflow compared to the no-bead overnight digestion (which has previously been shown³⁷ for SP3 carboxylate bead workflows).

Interestingly, while the peptide GRAVY score distributions of the rapid bead and overnight no-bead workflows were similar, the bead workflow appears to extract hydrophilic peptides to a greater extent than without beads (FIG. 9, panel b)). This difference is due to the presence of beads and is not resulting from the n-butanol extraction, as similar increases in hydrophilic peptide intensities were observed when comparing n-butanol extractions with and without beads (data not shown). The difference appears to be largely driven by the unique peptides identified in the bead workflow versus the non-bead workflow: peptides solely identified with SP3 tended to be more hydrophilic, while those solely identified with in-solution digestion tended to be more hydrophobic. However, the overlap in protein identifications was high (˜80% between two replicates without match-between-runs and ˜95% with match-between-runs). Previous SP3 datasets^{33,51} display this bias toward hydrophilicity when using the traditional carboxylate-coated beads, and the present data demonstrate that this is also true with unmodified beads. Notably, all bead types that were tested showed similar bias toward hydrophilic peptides.

Final Streamlined Multi-omic Workflow for Mass Spectrometry Analysis: Overall, these improvements led to a significantly streamlined multi-omics sample preparation workflow (FIG. 2). The first step of the streamlined workflow described in this example is adding an n-butanol-based monophasic extraction solvent with unmodified magnetic beads to sample. After a brief vortex, samples are sonicated (as needed) and then incubated on ice for 5 minutes. The incubation period allows proteins to aggregate onto the beads, while metabolites and lipids remain in the supernatant. The unbound metabolites and lipids are then removed for further analysis. Buffer and trypsin are subsequently added for protein digestion at 60° C. for 40 minutes, and the resulting peptides are acidified, desalted, and dried. At the conclusion of the workflow, all three -omes can be analyzed by the instruments within the same day as preparation. This method, termed the BAMM sample preparation method, is simpler and more consolidated than classic methodologies, saving an average of 11 steps and 19 hours when compared to published workflows.

After developing the simplified and consolidated BAMM method, it was validated for multiple sample types and formats (cell pellets, biofluids, cell culture plates) for widespread use (FIGS. 3 and 4). Regardless of the sample type, the BAMM method generates metabolomics, lipidomics, and proteomics data of comparable depth as published multi-omics studies, but at a fraction of the required effort (FIG. 3). For plasma, yeast, murine adipocytes from culture plates, mouse brain, and human cell line pellets, 67, 152, 61, 90, 51 metabolites (respectively), 260, 139, 412, 430, 445 lipids (respectively), and 515, 3578, 4322, 5197, 5552 proteins (respectively) were identified (on average, three technical replicates). Though the metabolite yields achieved with the BAMM method may appear somewhat lower than can be achieved with other methods, the applied filters were strict in confident identification of metabolites (e.g., threshold of 80 for cosine similarity score). Overall, this approach is highly versatile and will generate quality multi-omics data from any biological system or sample type.

Conclusion: The present example describes a simple and consolidated method (named BAMM) to prepare metabolites, lipids, and proteins from a single sample. This method combines an n-butanol-based monophasic extraction with paramagnetic bead technology, expediting small molecule extraction and protein digestion. This new strategy produces quality multi-omics data comparable to classic methodologies at a fraction of the time and effort. Prepared metabolites, lipids, and peptides are ready for MS analysis in about 3 hours, compared to about a day on average for current workflows. It was also noted that due to the use of magnetic beads, this method is potentially more amenable to robotic automation and multi-well plate formats for increased throughput. Additionally, this BAMM sample preparation method is optimized for LC-MS analysis.

Furthermore, the components of this workflow can also be adapted as necessary and used individually. For example, after validating the monophasic solvent extraction, this method was applied to a large-scale COVID-19 study for fast lipidomics sample preparation.²⁵ It is envision that this expedient method or its individual components will be particularly beneficial for specific applications wherein turnaround time is an important consideration, such as clinical screening, iterative process optimization, rapid process analytics, and large sample screens. These benefits are amplified even further when pairing this streamlined multi-omics sample preparation with an integrated acquisition method such as MOST.¹⁴

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

When a group of materials, compositions, components or compounds is disclosed herein, it is understood that all individual members of those groups and all subgroups thereof are disclosed separately. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. Additionally, the end points in a given range are to be included within the range. In the disclosure and the claims, “and/or” means additionally or alternatively. Moreover, any use of a term in the singular also encompasses plural forms.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements.

One of ordinary skill in the art will appreciate that starting materials, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used herein for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art.

### REFERENCES

(1) Zhang, B.; Kuster, B. Proteomics Is Not an Island: Multi-Omics Integration Is the Key to Understanding Biological Systems. Molecular and Cellular Proteomics 2019, DOI: 10.1074/mcp.E119.001693.
(2) Brademan, D. R.; Miller, I. J.; Kwiecien, N. W.; Pagliarini, D. J.; Westphall, M. S.; Coon, J. J.; Shishkova, E. Argonaut: A Web Platform for Collaborative Multi-Omic Data Visualization and Exploration. Patterns 2020, 1 (7). DOI: 10.1016/j.patter.2020.100122.
(3) Krassowski, M.; Das, V.; Sahu, S. K.; Misra, B. B. State of the Field in Multi-Omics Research: From Computational Needs to Data Mining and Sharing. Frontiers in Genetics 2020. DOI: 10.3389/fgene.2020.610798.
(4) Hebert, A. S.; Richards, A. L.; Bailey, D. J.; Ulbrich, A.; Coughlin, E. E.; Westphall, M. S.; Coon, J. J. The One Hour Yeast Proteome. Molecular and Cellular Proteomics 2014, 13 (1), 339-347.
(5) Shishkova, E.; Hebert, A. S.; Westphall, M. S.; Coon, J. J. Ultra-High Pressure (>30,000 Psi) Packing of Capillary Columns Enhancing Depth of Shotgun Proteomic Analyses. Analytical Chemistry 2018, 90 (19), 11503-11508.
(6) Meier, F.; Geyer, P. E.; Virreira Winter, S.; Cox, J.; Mann, M. BoxCar Acquisition Method Enables Single-Shot Proteomics at a Depth of 10,000 Proteins in 100 Minutes. Nature Methods 2018, 15 (6), 440-448.
(7) Hebert, A. S.; Thöing, C.; Riley, N. M.; Kwiecien, N. W.; Shiskova, E.; Huguet, R.; Cardasis, H. L.; Kuehn, A.; Eliuk, S.; Zabrouskov, V.; Westphall, M. S.; McAlister, G. C.; Coon, J. J. Improved Precursor Characterization for Data-Dependent Mass Spectrometry. Analytical Chemistry 2018, 90 (3), 2333-2340.
(8) Meier, F.; Brunner, A. D.; Koch, S.; Koch, H.; Lubeck, M.; Krause, M.; Goedecke, N.; Decker, J.; Kosinski, T.; Park, M. A.; Bache, N.; Hoerning, O.; Cox, J.; Rather, O.; Mann, M. Online Parallel Accumulation-Serial Fragmentation (PASEF) with a Novel Trapped Ion Mobility Mass Spectrometer. Molecular and Cellular Proteomics 2018, 17 (12), 2534-2545.
(9) Kelstrup, C. D.; Bekker-Jensen, D. B.; Arrey, T. N.; Hogrebe, A.; Harder, A.; Olsen, J. v. Performance Evaluation of the Q Exactive HF-X for Shotgun Proteomics. Journal of Proteome Research 2018, 17 (1), 727-738.
(10) Senko, M. W.; Remes, P. M.; Canterbury, J. D.; Mathur, R.; Song, Q.; Eliuk, S. M.; Mullen, C.; Earley, L.; Hardman, M.; Blethrow, J. D.; Bui, H.; Specht, A.; Lange, O.; Denisov, E.; Makarov, A.; Horning, S.; Zabrouskov, V. Novel Parallelized Quadrupole/Linear Ion Trap/Orbitrap Tribrid Mass Spectrometer Improving Proteome Coverage and Peptide Identification Rates. Analytical Chemistry 2013, 85 (24), 11710-11714.
(11) Danne-Rasche, N.; Coman, C.; Ahrends, R. Nano-LC/NSI MS Refines Lipidomics by Enhancing Lipid Coverage, Measurement Sensitivity, and Linear Dynamic Range. Analytical Chemistry 2018, 90 (13), 8093-8101.
(12) Perez de Souza, L.; Alseekh, S.; Scossa, F.; Fernie, A. R. Ultra-High-Performance Liquid Chromatography High-Resolution Mass Spectrometry Variants for Metabolomics Research. Nature Methods 2021. 18 (7), 733-746.
(13) Hutchins, P. D.; Russell, J. D.; Coon, J. J. LipiDex: An Integrated Software Package for High-Confidence Lipid Identification. Cell Systems 2018, 6 (5), 621-625.e5.
(14) He, Y.; Rashan, E. H.; Linke, V.; Shishkova, E.; Hebert, A. S.; Jochem, A.; Westphall, M. S.; Pagliarini, D. J.; Overmyer, K. A.; Coon, J. J. Multi-Omic Single-Shot Technology for Integrated Proteome and Lipidome Analysis. Analytical Chemistry 2021, 93 (9), 4217-4222.
(15) Ding, J.; Blencowe, M.; Nghiem, T.; Ha, S. M.; Chen, Y. W.; Li, G.; Yang, X. Mergeomics 2.0: A Web Server for Multi-Omics Data Integration to Elucidate Disease Networks and Predict Therapeutics. Nucleic Acids Research 2021, 49 (W1), W375-W387.
(16) Rohart, F.; Gautier, B.; Singh, A.; Lê Cao, K. A. MixOmics: An R Package for 'omics Feature Selection and Multiple Data Integration. PLoS Computational Biology 2017, 13 (11). DOI: 10.1371/joumal.pcbi.1005752.
(17) Lê Cao, K. A.; González, I.; Déjean, S. IntegrOmics: An R Package to Unravel Relationships between Two Omics Datasets. Bioinformatics 2009, 25 (21), 2855-2856.
(18) Dugourd, A.; Kuppe, C.; Sciacovelli, M.; Gjerga, E.; Gabor, A.; Emdal, K. B.; Vieira, V.; Bekker-Jensen, D. B.; Kranz, J.; Bindels, Eric. M. J.; Costa, A. S. H.; Sousa, A.; Beltrao, P.; Rocha, M.; Olsen, J. v.; Frezza, C.; Kramann, R.; Saez-Rodriguez, J. Causal Integration of Multi-omics Data with Prior Knowledge to Generate Mechanistic Hypotheses. Molecular Systems Biology 2021, 17 (1). DOI: 10.15252/msb.20209730.
(19) Li, X.; Zhou, X.; Teng, T.; Fan, L.; Liu, X.; Xiang, Y.; Jiang, Y.; Xie, P.; Zhu, D. Multi-Omics Analysis of the Amygdala in a Rat Chronic Unpredictable Mild Stress Model of Depression. Neuroscience 2021, 463, 174-183.
(20) Xu, L.; Zhao, Q.; Luo, J.; Ma, W.; Jin, Y.; Li, C.; Hou, Y.; Feng, M.; Wang, Y.; Chen, J.; Zhao, J.; Zheng, Y.; Yu, D. Integration of Proteomics, Lipidomics, and Metabolomics Reveals Novel Metabolic Mechanisms Underlying N, N-Dimethylformamide Induced Hepatotoxicity. Ecotoxicology and Environmental Safety 2020, 205. DOI: 10.1016/j.ecoenv.2020.111166.
(21) Rampler, E.; Egger, D.; Schoeny, H.; Rusz, M.; Pacheco, M. P.; Marino, G.; Kasper, C.; Naegele, T.; Koellensperger, G. The Power of LC-MS Based Multiomics: Exploring Adipogenic Differentiation of Human Mesenchymal Stem/Stromal Cells. Molecules 2019, 24 (19). DOI: 10.3390/molecules24193615.
(22) Coman, C.; Solari, F. A.; Hentschel, A.; Sickmann, A.; Zahedi, R. P.; Ahrends, R. Simultaneous Metabolite, Protein, Lipid Extraction (SIMPLEX): A Combinatorial Multimolecular Omics Approach for Systems Biology. Molecular and Cellular Proteomics 2016, 15 (4), 1453-1466.
(23) Nakayasu, E. S.; Nicora, C. D.; Sims, A. C.; Burnum-Johnson, K. E.; Kim, Y.-M.; Kyle, J. E.; Matzke, M. M.; Shukla, A. K.; Chu, R. K.; Schepmoes, A. A.; Jacobs, J. M.; Baric, R. S.; Webb-Robertson, B.-J.; Smith, R. D.; Metz, T. O. MPLEx: A Robust and Universal Protocol for Single-Sample Integrative Proteomic, Metabolomic, and Lipidomic Analyses. mSystems 2016, 1 (3). DOI: 10.1128/msystems.00043-16.
(24) Stefely, J. A.; Kwiecien, N. W.; Freiberger, E. C.; Richards, A. L.; Jochem, A.; Rush, M. J. P.; Ulbrich, A.; Robinson, K. P.; Hutchins, P. D.; Veling, M. T.; Guo, X.; Kemmerer, Z. A.; Connors, K. J.; Trujillo, E. A.; Sokol, J.; Marx, H.; Westphall, M. S.; Hebert, A. S.; Pagliarini, D. J.; Coon, J. J. Mitochondrial Protein Functions Elucidated by Multi-Omic Mass Spectrometry Profiling. Nature Biotechnology 2016, 34 (11), 1191-1197.
(25) Overmyer, K. A.; Shishkova, E.; Miller, I. J.; Balnis, J.; Bernstein, M. N.; Peters-Clarke, T. M.; Meyer, J. G.; Quan, Q.; Muehlbauer, L. K.; Trujillo, E. A.; He, Y.; Chopra, A.; Chieng, H. C.; Tiwari, A.; Judson, M. A.; Paulson, B.; Brademan, D. R.; Zhu, Y.; Serrano, L. R.; Linke, V.; Drake, L. A.; Adam, A. P.; Schwartz, B. S.; Singer, H. A.; Swanson, S.; Mosher, D. F.; Stewart, R.; Coon, J. J.; Jaitovich, A. Large-Scale Multi-Omic Analysis of COVID-19 Severity. Cell Systems 2021, 12 (1), 23-40.e7.
(26) Kang, J.; David, L.; Li, Y.; Cang, J.; Chen, S. Three-in-One Simultaneous Extraction of Proteins, Metabolites and Lipids for Multi-Omics. Frontiers in Genetics 2021, 12. DOI: 10.3389/fgene.2021.635971.
(27) Lapointe, C. P.; Stefely, J. A.; Jochem, A.; Hutchins, P. D.; Wilson, G. M.; Kwiecien, N. W.; Coon, J. J.; Wickens, M.; Pagliarini, D. J. Multi-Omics Reveal Specific Targets of the RNA-Binding Protein Puf3p and Its Orchestration of Mitochondrial Biogenesis. Cell Systems 2018, 6 (1), 125-135.e6.
(28) Overmyer, K.; Rhoads, T.; Merrill, A.; Ye, Z.; Westphall, M.; Acharya, A.; Shukla, S.; Coon, J. Proteomics, Lipidomics, Metabolomics and 16S DNA Sequencing of Dental Plaque from Patients with Diabetes and Periodontal Disease. Molecular and Cellular Proteomics 2021, 20. DOI: 10.1016/j.mcpro.2021.100126.
(29) Matyash, V.; Liebisch, G.; Kurzchalia, T. v.; Shevchenko, A.; Schwudke, D. Lipid Extraction by Methyl-Tert-Butyl Ether for High-Throughput Lipidomics. Journal of Lipid Research 2008, 49 (5), 1137-1146.
(30) Folch, J.; Lees, M.; Sloane Stanley, G. H. A simple method for the isolation and purification of total lipides from animal tissues. Journal Biological Chemistry 1957, 226 (1), 497-509.
(31) Bligh, E.; Dyer, W. A rapid method of total lipid extraction and purification. The Canadian Journal of Biochemistry and Physiology 1959, 37 (8), 911-917.
(32) König, G.; Reetz, M. T.; Thiel, W. 1-Butanol as a Solvent for Efficient Extraction of Polar Compounds from Aqueous Medium: Theoretical and Practical Aspects. Journal of Physical Chemistry B 2018, 122 (27), 6975-6988.
(33) Hughes, C. S.; Foehr, S.; Garfield, D. A.; Furlong, E. E.; Steinmetz, L. M.; Krijgsveld, J. Ultrasensitive Proteome Analysis Using Paramagnetic Bead Technology. Molecular Systems Biology 2014, 10 (10), 757.
(34) Virant-Klun, I.; Leicht, S.; Hughes, C.; Krijgsveld, J. Identification of Maturation-Specific Proteins by Single-Cell Proteomics of Human Oocytes. Molecular and Cellular Proteomics 2016, 15 (8), 2616-2627.
(35) Moggridge, S.; Sorensen, P. H.; Morin, G. B.; Hughes, C. S. Extending the Compatibility of the SP3 Paramagnetic Bead Processing Approach for Proteomics. Journal of Proteome Research 2018, 17 (4), 1730-1740.
(36) Hughes, C. S.; Moggridge, S.; Müller, T.; Sorensen, P. H.; Morin, G. B.; Krijgsveld, J. Single-Pot, Solid-Phase-Enhanced Sample Preparation for Proteomics Experiments. Nature Protocols 2019, 14 (1), 68-85.
(37) Batth, T. S.; Tollenaere, M. A. X.; Ruther, P.; Gonzalez-Franquesa, A.; Prabhakar, B. S.; Bekker-Jensen, S.; Deshmukh, A. S.; Olsen, J. v. Protein Aggregation Capture on Microparticles Enables Multipurpose Proteomics Sample Preparation. Molecular and Cellular Proteomics 2019, 18 (5), 1027-1035.
(38) Mori, H.; Dugan, C. E.; Nishii, A.; Benchamana, A.; Li, Z.; Cadenhead, T. S.; Das, A. K.; Evans, C. R.; Overmyer, K. A.; Romanelli, S. M.; Peterson, S. K.; Bagchi, D. P.; Corsa, C. A; Hardij, J.; Learman, B. S.; el Azzouny, M.; Coon, J. J.; Inoki, K.; MacDougald, O. A. The Molecular and Metabolic Program by Which White Adipocytes Adapt to Cool Physiologic Temperatures. PLoS Biology 2021, 19 (5). DOI: 10.1371/joumal.pbio.3000988.
(39) Lorenz, M. A.; Burant, C. F.; Kennedy, R. T. Reducing Time and Increasing Sensitivity in Sample Preparation for Adherent Mammalian Cell Metabolomics. Analytical Chemistry 2011, 83 (9), 3406-3414.
(40) Sambrook, J.; Russell, D. W. Fragmentation of DNA by Sonication. Cold Spring Harbor Protocols 2017, 2006 (23). DOI: 10.1101/pdb.prot4538.
(41) Cox, J.; Mann, M. MaxQuant Enables High Peptide Identification Rates, Individualized p.p.b.-Range Mass Accuracies and Proteome-Wide Protein Quantification. Nature Biotechnology 2008, 26 (12), 1367-1372.
(42) Cox, J.; Neuhauser, N.; Michalski, A.; Scheltema, R. A.; Olsen, J. v.; Mann, M. Andromeda: A Peptide Search Engine Integrated into the MaxQuant Environment. Journal of Proteome Research 2011, 10 (4), 1794-1805.
(43) Rgen Cox, J.; Hein, M. Y.; Luber, C. A.; Paron, I.; Nagaraj, N.; Mann, M. Accurate Proteome-Wide Label-Free Quantification by Delayed Normalization and Maximal Peptide Ratio Extraction, Termed MaxLFQ. Molecular & Cellular Proteomics 2014, 13, 2513-2526.
(44) Löfgren, L.; Forsberg, G. B.; Ståhlman, M. The BUME Method: A New Rapid and Simple Chloroform-Free Method for Total Lipid Extraction of Animal Tissue. Scientific Reports 2016, 6. DOI: 10.1038/srep27688.
(45) Teng, Q.; Huang, W.; Collette, T. W.; Ekman, D. R.; Tan, C. A Direct Cell Quenching Method for Cell-Culture Based Metabolomics. Metabolomics 2009, 5 (2), 199-208.
(46) Sitnikov, D. G.; Monnin, C. S.; Vuckovic, D. Systematic Assessment of Seven Solvent and Solid-Phase Extraction Methods for Metabolomics Analysis of Human Plasma by LC-MS. Scientific Reports 2016, 6. DOI: 10.1038/srep38885.
(47) Liu, Z.; Rochfort, S.; Cocks, B. G. Optimization of a Single Phase Method for Lipid Extraction from Milk. Journal of Chromatography A 2016, 1458, 145-149.
(48) Rabinowitz, J. D.; Kimball, E. Acidic Acetonitrile for Cellular Metabolome Extraction from Escherichia Coli. Analytical Chemistry 2007, 79 (16), 6167-6173.
(49) Kyte, J.; Doolittle, R. F. A Simple Method for Displaying the Hydropathic Character of a Protein. Journal of Molecular Biology 1982, 157 (1), 105-132.
(50) Bag, S.; Rauwolf, S.; Suyetin, M.; Schwaminger, S. P.; Wenzel, W.; Berensmeier, S. Buffer Influence on the Amino Acid Silica Interaction. ChemPhysChem 2020, 21 (20), 2347-2356.
(51) Gutierrez, D. B.; Gant-Branum, R. L.; Romer, C. E.; Farrow, M. A.; Allen, J. L.; Dahal, N.; Nei, Y. W.; Codreanu, S. G.; Jordan, A. T.; Palmer, L. D.; Sherrod, S. D.; McLean, J. A.; Skaar, E. P.; Norris, J. L.; Caprioli, R. M. An Integrated, High-Throughput Strategy for Multiomic Systems Level Analysis. Journal of Proteome Research 2018, 17 (10), 3396-3408.

1. A method for extracting biomolecules from a sample comprising the steps of:
a) mixing the sample with an extraction solvent and a plurality of immobilizing beads, wherein the extraction solvent is able to solubilize a first portion of biomolecules comprising lipids, carbohydrates, metabolites, and combinations thereof, and wherein the plurality of immobilizing beads are able to bind and immobilize a second portion of biomolecules comprising nucleic acids, proteins, polypeptides, and combinations thereof,
thereby generating an extraction solution comprising the first portion of biomolecules and generating bound immobilizing beads attached to the second portion of biomolecules;
b) separating the bound immobilizing beads from the extraction solution comprising the first portion of biomolecules;
c) separating the first portion of biomolecules from the extraction solution, thereby generating at least a first set of extracted biomolecules, and separating the second portion of biomolecules from the bound immobilizing beads, thereby generating at least a second set of extracted biomolecules.

2. The method of claim 1 wherein the extraction solution is a monophasic solution.

3. The method of claim 1 wherein the first portion of biomolecules comprises a mixture of lipids and metabolites, and the second portion of biomolecules comprises proteins, polypeptides, and combinations thereof.

4. The method of claim 3 further comprising digesting the proteins and polypeptides attached to the bound immobilizing beads.

5. The method of claim 4 wherein digesting comprises mixing the bound immobilizing beads attached to the proteins, polypeptides, and combinations thereof, with a protein digestion enzyme or chemical agent for a digestion time period between 30 minutes and 60 minutes at a digestion temperature between 40° C. and 80° C.

6. The method of claim 5 wherein the digestion time period is between 35 minutes and 50 minutes.

7. The method of claim 5 wherein the digestion temperature is between 55° C. and 65° C.

8. The method of claim 4 wherein digesting comprises mixing the bound immobilizing beads attached to the proteins, polypeptides, and combinations thereof, with a protein digestion enzyme or chemical agent for a digestion time period between 35 minutes and 45 minutes at a digestion temperature between 55° C. and 65° C.

9. The method of claim 1 wherein mixing the sample with the extraction solvent and the plurality of immobilizing beads comprises incubating the sample with the extraction solvent and the plurality of immobilizing beads for an incubation time period between 5 minutes and 1 hour.

10. The method of claim 1 wherein steps a) through c) are performed within three hours or less.

11. The method of claim 1 wherein the extraction solvent comprises, by volume, between 20% and 80% of n-butanol.

12. The method of claim 1 wherein the extraction solvent comprises, by volume, between 55%-65% n-butanol, between 15%-25% acetonitrile, and between 15%-25% water.

13. The method of claim 1 wherein the plurality of immobilizing beads are magnetic or paramagnetic beads.

14. The method of claim 1 wherein the plurality of immobilizing beads are unmodified silica beads.

15. The method of claim 19 comprising performing mass spectrometry analysis on the first set and second set of extracted biomolecules.

16. The method of claim 1 wherein the sample is a whole cell lysate.

17. The method of claim 1 wherein the first portion of biomolecules comprises a lipidome and metabolome of a cell, and the second portion of biomolecules comprises a proteome of the cell.

18. A method for extracting biomolecules from a sample comprising the steps of:
a) mixing the sample with an extraction solvent and a plurality of unmodified immobilizing beads, wherein the extraction solvent comprises, by volume, between 20% and 80% of n-butanol and is able to solubilize a first portion of biomolecules comprising lipids and metabolites, and wherein the plurality of unmodified immobilizing beads are able to bind and immobilize a second portion of biomolecules comprising proteins and polypeptides,
thereby generating a monophasic extraction solution comprising the first portion of biomolecules and generating bound immobilizing beads attached to the second portion of biomolecules;
b) separating the bound immobilizing beads attached to the second portion of biomolecules from the extraction solution comprising the first portion of biomolecules;
c) mixing the bound immobilizing beads attached to the proteins and polypeptides with a protein digestion enzyme or chemical agent for a digestion time period between 35 minutes and 45 minutes at a digestion temperature between 55° C. and 65° C.; and
d) separating the first portion of biomolecules from the extraction solution, thereby generating at least a first set of extracted biomolecules, and separating the second portion of biomolecules from the bound immobilizing beads, thereby generating at least a second set of extracted biomolecules,
wherein steps a) through d) are performed within three hours or less.

19. A kit for extracting biomolecules from a sample, said kit comprising:
a) an extraction solvent able to at least partially solubilize lipids, carbohydrates, biological metabolites, and combinations thereof,
b) a plurality of immobilizing beads able to bind and immobilize polypeptides, and
c) a digestion solution able to digest polypeptides, where the digestion solution comprises a protein digestion enzyme and/or a chemical agent.

20. The kit of claim 19 wherein the extraction solvent comprises 20-80% by volume n-butanol and one or more co-solvents selected from the group consisting of methanol, ethanol, water, acetone, and acetonitrile; the plurality of immobilizing beads comprise magnetic beads, paramagnetic beads, or unmodified silica beads; and the digestion solution comprises trypsin.
